(19) United States
(12) Patent Application Publication
(10) Pub. No.: US 2025/0207180 A1
(43) Pub. Date: Jun. 26, 2025

(54) SPATIALLY RESOLVED CYCLIC MULTIPLEXED DETECTION OF NUCLEIC ACIDS AND PROTEINS IN TISSUE

(71) Applicant: Akoya Biosciences, Inc., Menlo Park, CA (US)

(72) Inventors: Lydia Hernandez, Menlo Park, CA (US); Timothy B. Karpishin, Menlo Park, CA (US); Peter J. Miller, Marlborough, MA (US)

(21) Appl. No.: 19/000,124

(22) Filed: Dec. 23, 2024

Related U.S. Application Data
(60) Provisional application No. 63/613,739, filed on Dec. 21, 2023.

(51) Int. Cl. C12Q 1/6823; C12Q 1/28; C12Q 1/6804; C12Q 1/6876; G01N 21/64

(52) U.S. Cl. CPC C12Q 1/6823; C12Q 1/28; C12Q 1/6804; C12Q 1/6876; G01N 21/6456; G01N 2333/908

(57) ABSTRACT

Methods for detecting RNA in a sample include contacting the sample with a probe featuring a binding oligonucleotide sequence that hybridizes to at least a portion of an RNA in the sample and a probe identification oligonucleotide sequence associated with the portion of the RNA to which the probe hybridizes, contacting the sample with a catalytic reporter, contacting the sample with an anchoring reporter featuring a substrate conjugated to at least one barcode oligonucleotide sequence, where the substrate reacts with the catalytic moiety to deposit the anchoring reporter or a derivative thereof comprising the at least one barcode oligonucleotide sequence in the sample at a location in proximity to the probe, and contacting the sample with a reporter molecule.

### CROSS-REFERENCE TO RELATED APPLICATIONS

This application claims priority to U.S. Provisional Patent Application No. 63/613,739, filed on Dec. 21, 2023, the entire contents of which are incorporated herein by reference.

### TECHNICAL FIELD

This disclosure relates to methods, systems, and reagents for detection of target analytes including, but not limited to, RNA and proteins in biological samples, including tissue samples.

### BACKGROUND

Detection of expressed RNA sequences in tissue samples with cellular or sub-cellular spatial resolution can provide valuable information about the type, activity, and molecular processes of individual cells, including their interactions with one another and the tissue environment.

### SUMMARY

This disclosure features methods and systems for multiplexed imaging of biological samples, and reagents and kits to facilitate performing various steps of the methods. The methods involve, in part, depositing a plurality of substrate-oligonucleotide sequences associated with a specific analyte (i.e., a specific RNA sequence, protein, peptide, etc.) in the sample in proximity to the analyte, and then detecting the deposited substrate-oligonucleotide sequences by introducing reporter molecules that bind selectively to the substrate-oligonucleotide sequences.

The probe, amplification, and detection reagents described herein can be used for fluorescent multiplexed RNA detection in a cell or tissue sample using sequential readout of reporters. This provides for detection of more RNA targets than the number of available, optically distinct dyes. As a result, a larger number of RNA targets can be detected than would otherwise be possible based on the number of optically distinct dyes that satisfy spectral range and crosstalk limitations.

The disclosure provides for background removal by imaging the sample with substantially no RNA-associated dyes present, then correcting an image of the sample with RNA dyes present by removing the background signal without dyes present from each pixel.

In some embodiments, the methods described herein provide for amplified detection of analytes such as RNA molecules, proteins, peptides, and other species. To facilitate amplified detection, multiple fluorescent dye molecules are introduced for each RNA molecule. This disclosure features several different methods for implementing amplified detection.

In some embodiments, the disclosure provides methods for RNA detection where a sample is prepared for RNA staining and RNA probes are deposited where each RNA probe includes a probe ID nucleotide sequence. Enzyme reporters bind to the probe ID sequence of the RNA probes and catalyze covalent binding of substrate-oligonucleotide (“substrate-oligo”) molecules to the sample at regions corresponding to the RNA probes. These remain durably bound to the sample even if subjected to elevated temperatures or other conditions that would dehybridize or damage the RNA probes.

In some embodiments, the disclosure provides methods for detecting multiple RNA types by introducing multiple RNA probe species into the sample, each probe species including multiple probe ID sequences, and depositing multiple substrate-oligo species, each of which contains a unique barcode ID sequence. Deposition is done in cycles by introducing an enzyme reporter species that targets a particular RNA probe ID species, localizing it to the associated RNA probe molecules, and introducing a substrate-oligo species with a unique barcode ID. That substrate-oligo is deposited at locations in the sample corresponding to RNA probes bearing the probe ID associated with that cycle, after which unbound molecules of that species of substrate-oligo are removed by washing steps. Then the enzyme reporters previously introduced into the sample are inactivated or removed prior to the next cycle of deposition. Each cycle involves a single enzyme reporter species targeting a single RNA probe ID sequence and then depositing a single species of substrate-oligo with a unique barcode ID. This process is repeated until substrate-oligo species have been deposited for each RNA probe ID species of interest.

The steps of the various workflows described herein can be performed manually, or certain steps (or even entire workflows) can be performed in automated fashion by automated staining systems. Suitable systems for performing the described steps/workflows are available commercially and will be described in greater detail below. The various steps and workflows, whether performed manually or in automated fashion (or a combination thereof), can be applied to interrogate multiple samples sequentially or in parallel.

Once the sample contains deposited substrate-oligo species bound to the sample at locations corresponding to RNA probes having the corresponding RNA probe ID, reporter molecules are introduced and bind to the RNA probe ID sequences. The reporter molecules typically include an oligonucleotide sequence conjugated to one or more dye molecules (e.g., fluorescent dye molecules). Optical signals (e.g., fluorescence emission) from the dye molecules is detected and used to form an image of the RNA species in the sample. The RNA probes optionally can be removed from the sample at this point by heating, washing, use of denaturants such as dimethylsulfoxide (DMSO) or urea, or a combination of these.

The nucleotide sequence of the reporter molecules recognizes a specific barcode ID sequence of a substrate-oligo deposited in the sample. The reporter molecules therefore localize in the sample at positions corresponding to substrate-oligo molecules having that barcode ID. An image of the dye is acquired, after which either the dye or the entire reporter molecule is removed, or the dye is inactivated. The methods described herein provide for imaging many substrate-oligo species, and thereby many RNA probes, by repeating this process until reporter molecules have been imaged for each substrate-oligo species for which detection is desired. Several species of reporter molecules can be introduced and imaged in each cycle by choosing several dyes that are optically distinct and assigning distinct dyes to different reporter molecule molecules. Thus, the number of cycles that are performed to detect all RNA target species in the sample can be lower than the number of reporter molecules based on the number of distinct dyes employed in each imaging cycle.

In addition to sequential imaging of many RNA species, another aspect of the disclosure provides for repeatedly imaging some or all species when that is desired. The disclosure provides for detecting RNA by sequential imaging of substrate-oligo molecules associated with the RNA, which are durably bound to the sample. By dehybridizing reporter molecules from the substrate-oligo molecules after imaging, those substrate-oligo molecules become available for detection once again and the sample can be imaged again by re-introducing reporting molecules that selectively bind to barcode ID sequences of substrate-oligo species of interest, and acquiring one or more images of the sample. This flexibility can be advantageous, for example, if the imaging apparatus or software fails to obtain a satisfactory sample image in the first attempt. The disclosure provides for re-imaging the sample if that occurs, with little or no loss of information or sensitivity.

Imaging steps can be performed on a microscope with a fluidics delivery system to provide reporter molecules, perform wash steps, and dehybridize reporter molecules automatically. The steps can be performed using systems like the PhenoCycler Fusion™ (available from Akoya Biosciences, Menlo Park, CA) but other systems can also be used. Steps described herein can also be performed manually. For example, a technician can attach a coverslip to the sample after reporter molecules are localized at locations corresponding to bound substrate-oligo molecules (with barcode IDs as part of the oligonucleotide sequence), manually obtain an image of the sample with a microscope, remove the coverslip, and proceed to the next cycle of reporter molecule introduction.

Images from successive imaging cycles can be registered by introducing 4′,6-diamidino-2-phenylindole (DAPI) along with the reporter molecules, and obtaining an image in which optical signals due to DAPI are detected. Images based on DAPI signals can then be used to detect and correct for pixel shifts between cycles.

In some embodiments, an image of the sample can be obtained with DAPI only and no reporter molecules present, to measure the sample fluorescence signal at each pixel location in the sample. Subtracting this signal from images corresponding to optical signals of the reporter molecules can improve RNA detection sensitivity and accuracy, and can reduce or eliminate undesirable optical signals arising from tissue fluorescence background signal (e.g., tissue autofluorescence). DAPI-only images can be obtained before the first reporter molecules are introduced, after all reporter molecules have been imaged, or once the reporter molecules have been removed or inactivated for any given cycle of imaging. Multiple DAPI-only images can be obtained for this purpose, from which an average or scaled pixel background signal value is obtained that is subtracted from each pixel location in the reporter molecule images.

The disclosure also provides methods for detection of RNA molecules and proteins, peptides, and other amino acid sequences in the same sample. For example, the methods described herein can be used to image multiple RNA target species and multiple proteins/peptides/amino acid sequences in a single tissue section using RNA probes, enzyme reagents, substrate-oligos, and reporter molecules, together with labeled antibodies that include oligonucleotide sequences having barcode ID sequences. In some embodiments, antibody staining is performed after substrate-oligo deposition (to detect RNA target molecules) is concluded. In certain embodiments, antibody staining is performed prior to deposition of substrate-oligos for RNA detection. Fixation can optionally be performed after antibody staining to cross-link the antibodies to the sample.

In some embodiments, a sample can be prepared with substrate-oligo molecules and oligonucleotide-labeled antibodies localized to regions on the sample associated with known RNA species and proteins respectively, and the sample can be imaged using reporter molecules that recognize barcode ID sequences in the substrate-oligo molecules and on the labeled antibodies. During imaging, RNA detection can be performed first via the reporter molecules that bind to the substrate-oligo barcode IDs, followed by protein detection via the reporter molecules that bind to barcodes ID sequences of the labeled antibodies. Alternatively, detection can be performed in the reverse order. Still further, as another alternative, one or more imaging cycles can be performed in which certain reporter molecules are introduced that bind to barcode ID sequences of substrate-oligos (e.g., to perform RNA target detection), and certain reporter molecules are introduced that bind to barcode ID sequences of labeled antibodies (e.g., to perform protein/peptide/amino acid sequence detection). Thus, in a single cycle, both RNA targets and protein/peptide/amino acid sequence targets can be detected.

In certain embodiments, a sample can be prepared with substrate-oligo molecules localized to regions in the sample associated with target RNA sequences, and imaged via reporter molecules that bind to barcode ID sequences of the substrate-oligo molecules. The reporter molecules are then removed, or dye molecules of the reporter molecules are removed or inactivated. Imaging of the RNA-associated reporter can be performed iteratively (e.g., in multiple cycles) to detect all target RNA analytes in the sample. One or more primary antibody probes, each of which binds to a different target protein/peptide/amino acid sequence can be incubated with the sample. Where the primary antibody probes are each conjugate to a different oligonucleotide barcode ID sequence, the primary antibody probes can be imaged by introducing reporter molecules that selectively bind to different barcode ID sequences. Alternatively, the primary antibody probes bound to target proteins/peptides/amino acid sequences in the sample can be incubated with labeled secondary antibody probes that localize at the locations of corresponding primary antibody probes by binding to the primary antibody probes. The labeled secondary antibody probes are conjugated to different barcode ID sequences. Target proteins/peptides/amino acids can then be detected by introducing reporter molecules that selectively bind to the different barcode ID sequences of the secondary antibody probes, and imaging the reporter molecules in the sample. Antibody probes (both primary and/or secondary antibody probes) can optionally be removed by heat or chemical means, and another cycle of antibody probe-based imaging can be performed. Imaging of reporter molecules associated with antibody probes can precede or follow the imaging of reporter molecules associated with substrate-oligos with barcode ID sequences that correspond to RNA target species.

The various steps of the methods described herein can be performed with a wide variety of different RNA probes including simple oligonucleotide sequences with an RNA recognition sequence and a probe ID sequence, paired probes where the pair each provide a portion of the probe ID sequence so both are typically present for an enzyme reporter to bind and localize there, and hybridized DNA structures that produce amplification by including multiple copies of the probe ID sequence to which enzyme reporters can bind.

While the methods described herein provide for direct detection where each RNA molecule is associated with a single probe ID sequence, complementary enzyme reporter, substrate-oligo species, and a single reporter molecule, the methods described also include implementations in which more complex associations with probe and/or barcode sequence IDs are used for detection of RNA and/or protein/peptide/amino acid targets. For example, in some embodiments, each RNA target analyte is associated with a plurality of probe IDs, enzyme reporter species, substrate oligo species, and reporter molecules in a combinatorial detection scheme. In combinatorial detection, each RNA target analyte is assigned a unique combination of probe IDs and the identity of the RNA species at each location in the sample is determined by identifying the combination of reporter molecules that are present at that location. Combinatorial detection schemes allow for the detection of many more RNA species N than the number of different reporter molecules M used for detection.

In some embodiments, the methods described herein provide for confirmational detection where each RNA target analyte is assigned a unique probe ID sequence along with a probe ID sequence that is shared by all RNA target analytes, and detection of an RNA target analyte at a location is confirmed when both a unique reporter molecule (complementary to the unique probe ID sequence) and a shared reporter molecule (complementary to the shared probe ID sequence) are present at that location in the sample. This enables improved specificity since false detection event are reduced or eliminated. When only one of the reporter molecules (e.g., only the unique reporter molecule or only the shared reporter molecule) are detected at the location, the detected signal can be discarded as it is assumed to be derived from a spurious binding event or localized concentration of a reporter molecule.

Some embodiments and/or methods described herein can be performed by software (executed on hardware), hardware, or a combination thereof. Hardware modules may include, for example, a general-purpose processor, a field programmable gate array (FPGA), and/or an application specific integrated circuit (ASIC). Software modules (executed on hardware) can be expressed in a variety of software languages (e.g., computer code), including C, C++, Java™, Ruby, Visual Basic™, and/or other object-oriented, procedural, or other programming language and development tools. Examples of computer code include, but are not limited to, micro-code or micro-instructions, machine instructions, such as produced by a compiler, code used to produce a web service, and files containing higher-level instructions that are executed by a computer using an interpreter. For example, embodiments may be implemented using imperative programming languages (e.g., C, Fortran, etc.), functional programming languages (Haskell, Erlang, etc.), logical programming languages (e.g., Prolog), object-oriented programming languages (e.g., Java, C++, etc.) or other suitable programming languages and/or development tools. Additional examples of computer code include, but are not limited to, control signals, encrypted code, and compressed code.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the subject matter herein, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments are set forth in the accompanying drawings and the description below. Other features and advantages will be apparent from the description, drawings, and claims.

### DESCRIPTION OF DRAWINGS

FIG. 1A is a schematic diagram showing an example of an RNA probe that is used to detect a target RNA analyte.

FIG. 1B is a schematic diagram showing two example RNA probes that are used to detect a target RNA analyte.

FIG. 1C is a schematic diagram showing two example RNA probes and an example preamplifier molecule that are used to detect a target RNA analyte.

FIG. 1D is a schematic diagram showing the example RNA probes and preamplifier molecule of FIG. 1C, and example amplifier molecules hybridized to the preamplifier molecule.

FIG. 2A is a schematic diagram showing an example of an enzymatic reporter molecule.

FIG. 2B is a schematic diagram showing an example of a substrate-oligonucleotide molecule.

FIG. 2C is a schematic diagram showing an example of a reporter molecule.

FIG. 2D is a schematic diagram showing an example of a labeled antibody (primary or secondary) probe.

FIG. 3A is a schematic diagram of a sample that includes multiple target RNA species to which RNA probe molecules are bound.

FIG. 3B is a schematic diagram of a sample that includes a target RNA species to which two RNA probe molecules are bound, an amplifier molecule bound to the two RNA probe molecules, and an enzyme reporter molecule bound to the amplifier molecule.

FIG. 3C is a schematic diagram showing the sample, RNA probe molecules, amplifier molecule, and enzyme reporter molecule of FIG. 3B, and multiple substrate-oligo molecules deposited in the sample.

FIG. 3D is a schematic diagram showing the sample, RNA probe molecules, amplifier molecule, enzyme reporter molecule, and substrate-oligo molecules of FIG. 3C, after inactivation of the enzyme reporter molecule.

FIG. 3E is a schematic diagram showing the sample, RNA probe molecules, amplifier molecule, enzyme reporter molecule, and substrate-oligo molecules of FIG. 3C, after cleavage of the enzyme from the enzyme reporter molecule.

FIG. 3F is a schematic diagram showing the sample, RNA probe molecules, amplifier molecule, enzyme reporter molecule, and substrate-oligo molecules of FIG. 3C, after dehybridization of the enzyme reporter molecule from the amplifier molecule.

FIG. 4 is a schematic diagram of a sample that includes different target RNA species to which two RNA probe molecules are bound, an amplifier molecule bound to the two RNA probe molecules, multiple enzyme reporter molecules bound to each amplifier molecule, and multiple substrate-oligo molecules deposited in the sample in the vicinity of each target RNA species.

FIG. 5A is a schematic diagram of a sample that includes different target RNA species, and multiple substrate-oligo molecules deposited in the sample in the vicinity of each target RNA species.

FIG. 5B is a schematic diagram of the sample of FIG. 5A, in which labeled antibody probes have been introduced to bind to target protein, peptide, and/or amino acid sequence targets in the sample.

FIG. 6A is a schematic diagram showing a sample with reporter molecules hybridized to substrate-oligo molecules bound to the sample at locations corresponding to a target RNA analyte.

FIG. 6B is a schematic diagram showing the sample of FIG. 6A after dehybridization of the reporter molecules.

FIG. 6C is a schematic diagram showing the sample of FIG. 6A after cleavage of dye molecules from the reporter molecules.

FIG. 6D is a schematic diagram showing the sample of FIG. 6A with labeled antibody probes bound to target protein, peptide, and/or amino acid sequence targets in the sample, and reporter molecules hybridized to the labeled antibody probes.

FIG. 6E is a schematic diagram showing the sample of FIG. 6D with the reporter molecules dehybridized from the labeled antibody probes.

FIG. 7 is a flow chart showing a series of example steps for detecting target RNA analytes in a sample.

FIG. 8A is a flow chart showing a series of example steps for detecting target RNA analytes and target protein, peptide, and/or amino acid sequences in a sample.

FIG. 8B is a flow chart showing another series of example steps for detecting target RNA analytes and target protein, peptide, and/or amino acid sequences in a sample.

FIG. 8C is a flow chart showing another series of example steps for detecting target RNA analytes and target protein, peptide, and/or amino acid sequences in a sample.

FIG. 9 is a flow chart showing a series of example steps for processing sample image information obtained from reporter molecule molecules in the sample.

FIG. 10 is a table showing examples of combinations of probe ID sequences that can be used to detect multiple target RNA analytes.

FIG. 11 is a flow chart showing a series of example steps for detecting a RNA target analyte using a combinatorial detection scheme.

FIG. 12 is a table showing examples of combinations of probe ID sequences that can be used to detect multiple target RNA analytes in a sample.

FIG. 13 is a flow chart showing another series of example steps for detecting a RNA target analyte using a confirmational detection scheme.

Like reference symbols in the various drawings indicate like elements.

### DETAILED DESCRIPTION

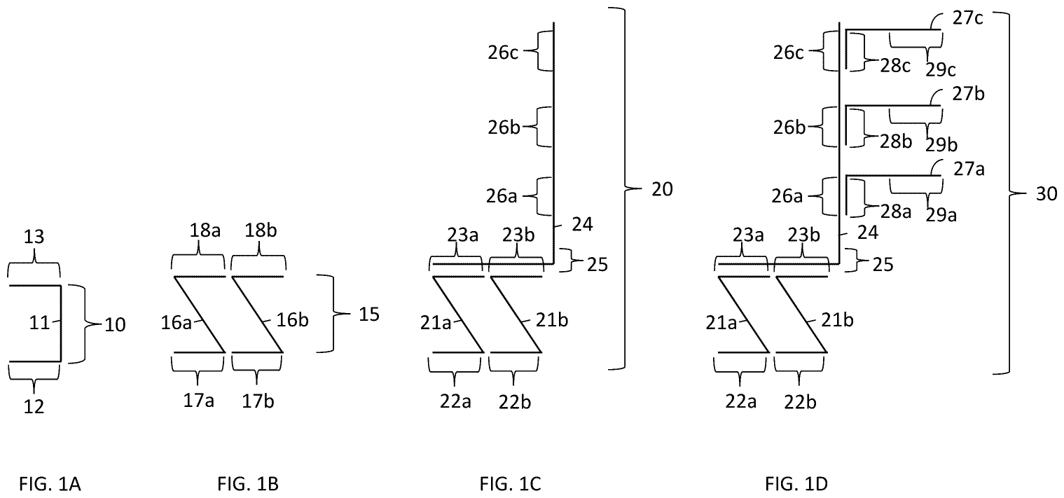

This disclosure features RNA probes that are used to recognize and selectively bind to specific RNA target analytes in a sample such as, but not limited to, cells and tissue samples. Examples of RNA probe molecules and other molecules that can be used for RNA analyte detection are shown in FIGS. 1A-1D.

FIG. 1 is a schematic diagram showing a RNA probe molecule 10 with an oligonucleotide sequence 11 that includes a target recognition sequence 12 and a probe ID sequence 13. Target recognition sequence 12 includes, largely, a reverse complement of base pairs to a target RNA analyte's sequence of bases.

In some embodiments, sequence 11 includes a DNA sequence, and thymine (T) in the target recognition sequence 12 is used to complement uracil (U) in the RNA sequence being targeted. Other nucleotides or synthetic nucleotides such as peptide nucleic acid (PNA) can also be used in sequence 11 if desired, and target recognition sequence 12 can include complementary synthetic nucleotides to those in the RNA.

The target recognition sequence 12 can be selected based on the known art of RNA probe design, and the specific RNA sequence and sequence length can be chosen to ensure that it is specific to the RNA target analyte of interest, without undue binding to other, non-targeted RNA molecules. The sequence length and content affect the thermodynamics of the binding interaction and are selected according to known principles of RNA probe design.

The probe 10 also comprises a probe ID sequence 13. This sequence is recognized by other reagents used in the disclosure that directly or indirectly couple the probe oligonucleotide sequence 11 to an enzyme reporter. It is used to associate a specific probe ID sequence with a given probe 10. Its length and sequence are designed to a desired affinity for the enzyme reporter. The affinity may be chosen according to the experiment protocol at hand, to assure that the enzyme reporters remain localized to the RNA molecule during the step of enzymatic deposition of substrate-oligo molecules on the sample.

In the example of FIG. 1A, a single probe molecule 10 binds to an RNA target analyte in the sample. However, a wide variety of different probes and associated molecules can be used to specifically bind to RNA target analytes. Probe molecules and other associated molecules, such as preamplifier molecules and amplifier molecules, can be used to form probe assemblies that contain one or more binding sites for enzyme reporter molecules.

FIG. 1B is a schematic diagram showing a probe assembly 15 that includes two probes, each of which includes an oligonucleotide sequence (16a, 16b). The oligonucleotide sequences include target recognition sequences 17a and 17b which recognize adjacent sequences in the RNA molecule, and probe ID sequences 18a and 18b. Taken together, sequences 18a and 18b form the sequence that will be coupled, directly or indirectly, to an enzymatic reporter molecule. This pairing provides improved specificity, since an individual off-target binding event by probe 16a or 16b does not contain the full sequence produced when both probes are present.

Accordingly, the design of probe ID sequences 18a and 18b is performed with the goal of providing a combined probe ID sequence that is unique relative to other probe ID sequences, not cross-reactive with other probe ID sequences, and provides the desired affinity for binding an enzymatic reporter molecule when paired but not when present singly as either 18a or 18b on its own.

FIG. 1C shows another example of a probe assembly 20. A pair of probes with oligonucleotide sequences 21a and 21b bind to a target RNA analyte. The sequences 21a and 21b include target recognition sequences 22a and 22b and probe ID sequences 23a and 23b, which create a combined probe ID sequence when both probes bind to the target RNA species. Preamplifier molecule 24 contains an oligonucleotide sequence that includes a probe ID recognition sequence 25 that recognizes the combined probe ID sequences 23a and 23b, along with one or more attachment sequence regions 26a-26c. The attachment sequence regions on the preamplifier molecule in this probe assembly act as the probe ID sequences for the overall probe assembly 20. The preamplifier molecule can, in some embodiments (and as shown in FIG. 1C), provide multiple copies of the probe ID sequence, so a plurality of enzyme reporter molecules can recognize and bind to it, thereby allowing a larger number of substrate-oligos to be deposited in the sample and amplifying measured optical signals associated with the RNA target analyte during sample imaging.

While the example preamplifier molecule 24 in FIG. 1C has three attachment sequence regions, more generally the preamplifier molecule 24, when used, can include as few as one attachment sequence region, or many such regions. For example, there may be 5 or more, 10 or more, 20 or more, 30 or more, 50 or more, 100 or more, or even more attachment sequence regions in preamplifier molecule 24. Use of more regions enables binding more enzyme reporter molecules per probe assembly, providing additional amplification. The number of regions can be selected based on a desired signal amplification during sample imaging.

FIG. 1D shows another type of a probe assembly 30 that includes two probes and a preamplifier molecule similar to the probe assembly in FIG. 1C. In addition, probe assembly 30 includes amplifier molecules 27a, 27b, and 27c. The amplifier molecules include oligonucleotide sequences with attachment sequence recognition regions 28a, 28b, and 28c that recognize and hybridize to attachment sequences 26a, 26b, and 26c on the preamplifier molecule, and probe ID sequences 29a, 29b, and 29c, respectively.

The amplifier molecules depicted in FIG. 1D can each have a single probe ID sequence (i.e., sequences 29a, 29b, and 29c can be the same). In some embodiments, probe assemblies of this type incorporate multiple probe ID sequences on each amplifier. Overall, there may be multiple amplifiers hybridized to the preamplifier, each of which has multiple probe ID sequences. This arrangement can provide a great number of probe ID sequences per probe assembly. For example, there may be 10 or more, or 20 or more, or 30 or more, or 40 or more, or 50 or more, or 80 or more, or 100 or more, or 150 or more, or 200 or more, or 300 or more, or 400 or more, or 500 or more probe ID sequences in the overall probe assembly. This type of probe assembly is capable of high amplification.

It should be noted that when amplifier molecules include multiple probe ID sequences, in some embodiments all such probe ID sequences are the same. However, in certain embodiments, some of the probe ID sequences may differ, e.g., in embodiments in which combinatorial and/or confirmation detection schemes are employed. Such detection schemes are discussed in greater detail subsequently.

Commercially available examples of the type of probe assemblies shown in FIG. 1D include View RNA™ probes (available from ThermoFisher Scientific, Waltham, MA) and RNAscope™ Multiplex v2 probes (available from Advanced Cell Diagnostics, Newark, CA).

In general, the methods described herein can be implemented with any type of probe that localizes enzyme reporter molecules in the sample in proximity to RNA analytes in a sufficient amount, and with enough specificity, to catalyze substrate-oligo molecule deposition to yield detectable signals. The decision to choose one probe type or another can be made based on factors such as cost, preparation time, ease of automation, and compatibility of sample pretreatment with protein detection.

Use of multiple probes to detect each RNA target analyte enables assignment of more than one probe ID to a given RNA analyte. The methods described herein can, in some embodiments, use a plurality of probe species targeting a single RNA analyte, with more than one probe ID represented among the multiple probe species. In such embodiments, one or more RNA target analytes can be bound to multiple probes, collectively having a plurality of probe IDs. In this way, that RNA analyte is associated with a plurality of known probe IDs.

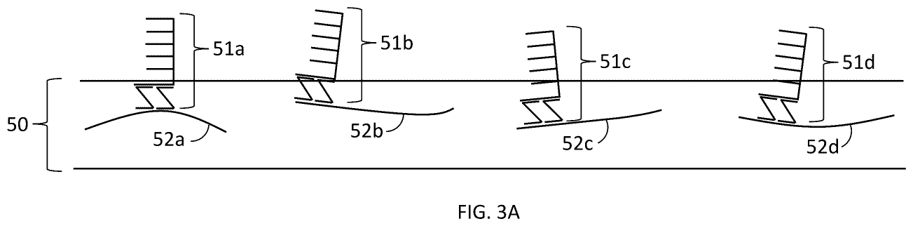

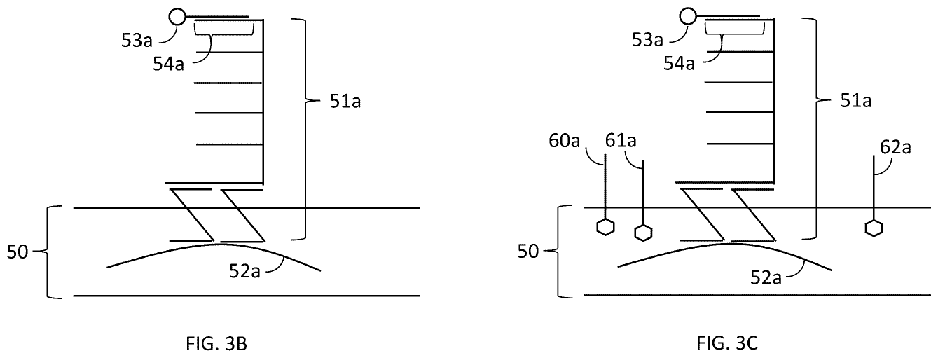

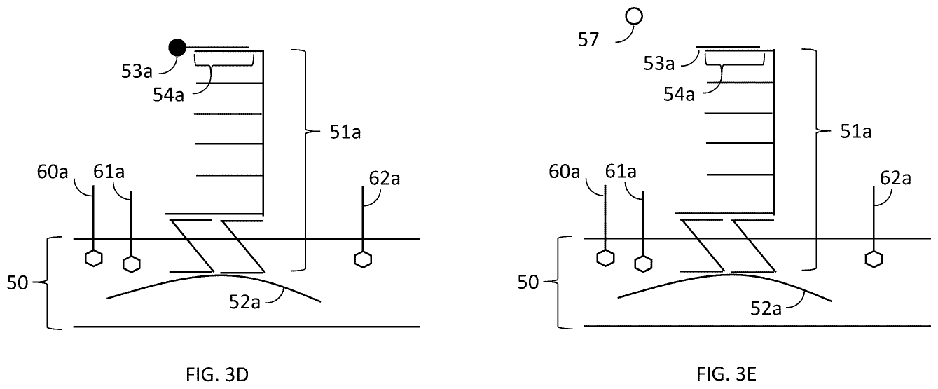

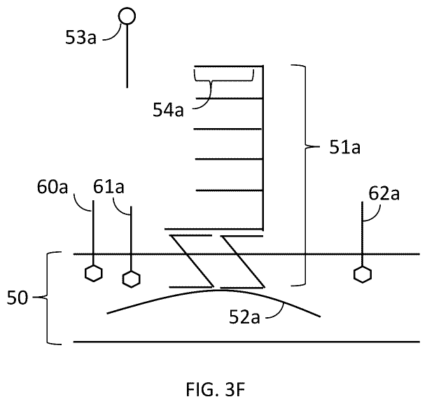

FIGS. 3A-3F are schematic diagrams showing a series of steps that can be performed to detect multiple RNA target analytes in a sample. In FIG. 3A, sample 50 includes multiple RNA target analytes 52a-52d. Probe assemblies 51a-51d are introduced into sample 50 by binding one or more probe molecules to each RNA target analyte, and optionally binding one or more preamplifier molecules and/or amplifier molecules to the bound probe molecules, as discussed above. Any of the various probe, preamplifier, and amplifier molecules described herein can be used to construct probe assemblies 51a-51d bound to the target analytes. In particular, any of the probes and probe assemblies shown in FIGS. 1A-1D can be used for this purpose.

In general, each probe or probe assembly introduced into sample 50 targets a different RNA analyte. This provides greater likelihood of detecting the RNA analyte in settings where the RNA molecule may be incomplete (so a given probe's target sequence may not be present), or where not all probes are able to access and recognize their sequence in the RNA molecule, or a variety of other factors are present that reduce the likelihood of any given probe successfully binding to the RNA molecule. Also, the use of multiple probes provides greater signal for detection. The design of multiple probes, and the decision to select distinct RNA sequences for the probes, or the use overlapping RNA sequences, is understood to those skilled in the art of RNA probe design.

In FIG. 3B, enzyme reporter molecules bind to the probe ID sequences of the probe assemblies. FIG. 3B only shows probe assembly 51a, but it should be understood that similar binding occurs for the other probe assemblies as well. Enzyme reporter molecule 53a hybridizes to probe ID sequence region 54a of probe assembly 51a bound to target RNA analyte 52a in sample 50.

Next, in FIG. 3C, the bound enzyme reporter molecules catalyze deposition of substrate-oligo molecules in the vicinity of the respective RNA target analytes. As in FIG. 3B, only probe assembly 51a and target RNA analyte 52a are shown in FIG. 3C for clarity, but similar deposition occurs for all RNA analytes shown in FIG. 3A. Specifically, substrate-oligo molecules 60a-62a are activated by enzyme reporter molecule 53a and are deposited in sample 50 near to the location of RNA target analyte 52a. In some embodiments, substrate-oligo molecules 60a-62a are the same. In certain embodiments, however, substrate-oligo molecules 60a-62a may differ, e.g., when combinatorial and/or confirmational detection schemes are used.

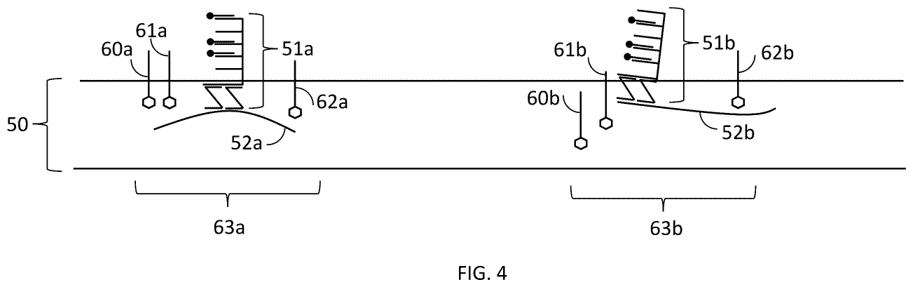

FIG. 4 is a schematic diagram showing the sample of FIG. 3A, and RNA target analytes 52a and 52b in the sample. Probe assemblies 51a and 51b are bound to target analytes 52a and 52b at regions 63a and 63b of the sample, respectively. In FIG. 4, multiple enzyme reporter molecules are bound to each probe assembly. The enzyme reporter molecules bound to probe assembly 5la catalyze deposition of substrate-oligos 60a-62a in region 63a, and the enzyme reporter molecules bound to probe assembly 51b catalyze deposition of substrate-oligos 60b-62b in region 63b.

FIGS. 3D-3F show various methods by which enzyme reporter molecules are removed or inactivated in sample 50 after substrate-oligos have been deposited. In FIG. 3D, for example, the enzyme in enzyme reporter molecule 53a is chemically inactivated. In FIG. 3E, the enzyme is cleaved from the enzyme reporter molecule 53. In FIG. 3F, the enzyme reporter molecule 53 is dehybridized from the preamplifier molecule.

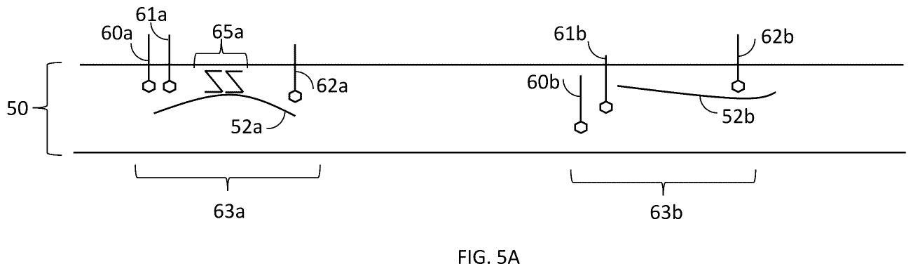

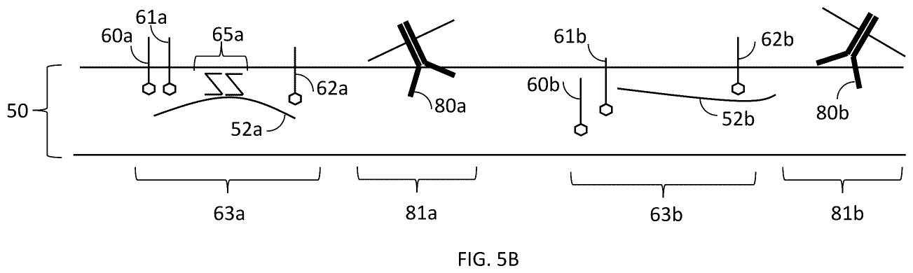

FIG. 5A shows the sample of FIG. 4 after removal of a portion or all of the probe assembly bound to the RNA target analytes of interest. A portion of the probe assembly 51a bound to analyte 52a (e.g., the preamplifier molecule) has been removed via dehybridization. Probes 65a remain hybridized to analyte 52, and substrate-oligos 60a-62a remain durably bound to the sample in region 63a. The entire probe assembly 51b bound to analyte 52b has been removed from sample 50, leaving only substrate-oligos 60b-62b durably bound to sample 50 in region 63b.

The methods described herein are used to detect multiple RNA target analytes in a sample. In some embodiments, the same type of probe assembly (with each assembly associating different probe ID sequences for each RNA target analyte) is used. In certain embodiments, different types of probe assemblies can be used to target different RNA analytes. In some embodiments, different types of probe assemblies can be used to target the same RNA analyte, although generally even when different types of probe assemblies are used, the same probe ID sequence (or combination of probe ID sequences) is associated with each target RNA analyte.

In the methods described herein, unique and orthogonal probe ID sequences are employed and associated with each RNA target analyte so that an enzyme reporter molecule intended for a given probe ID sequence does not bind to other probe ID sequences.

Development of multiple, unique probe ID sequences not prone to cross-reaction in this way is known in the art of probe design. It can be done in-silico based on computational methods, or by testing candidate probe ID sequences with the recognition sequences for other probe IDs and measuring cross-reaction, or by a combination of these techniques.

For example, the sequences listed in U.S. Pat. No. 10,370,698 as SEQ IDs 48-94 can be used as probe ID sequences in conjunction with the sequences listed as SEQ IDs 1-47 as probe ID recognition sequences. These exhibit melting temperatures (Tm) in the range 35° C.-50° C. The entire contents of U.S. Pat. No. 10,370,698 are incorporated herein by reference.

In some embodiments, multiple RNA target analytes are detected in a sample, with multiple probe ID sequences assigned to each RNA analyte. A given probe ID sequence may be assigned to multiple RNA analytes, but the combination of RNA probe ID sequences is unique for each RNA analyte.

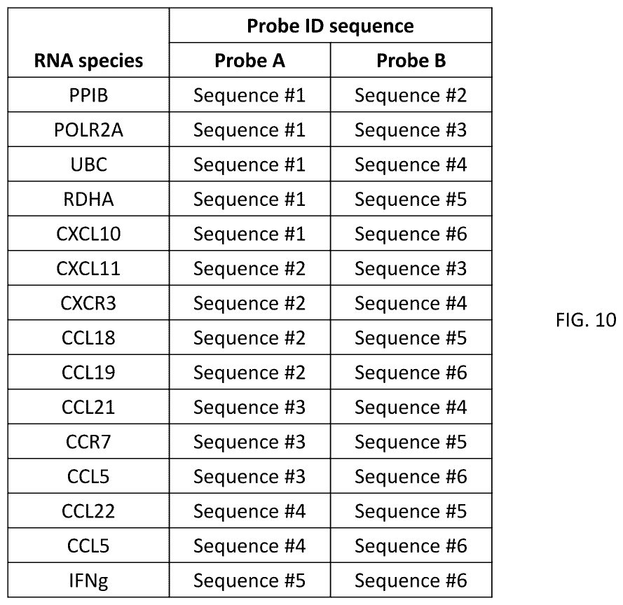

An example of using combinations of probe ID sequences to uniquely identify different RNA analytes is illustrated in FIG. 10. In FIG. 10, six unique probe ID sequences are used in combinations of two sequences assigned to each RNA target analyte to uniquely identify 15 different analytes combinatorially. In general for such combinatorial detection schemes, more RNA analytes M than the number of unique probe ID sequences N used for detection can be identified in such a combinatorial detection scheme.

If each RNA analyte is assigned K probe ID sequences, there are

$$M = ( \begin{matrix}N \\ K\end{matrix} ) = \frac{N !}{K ! ( N - K ) !}$$

unique combinations. In the example of FIG. 10, with 6 probe ID sequences and 2 probes per RNA species, a combinatorial detection system can detect 15 RNA analytes. In practical terms, N sets the number of enzymatic reporter species, substrate-oligo species, and reporter molecule species, as well as the number of enzymatic deposition cycles. Reducing N for a given number of RNA species M means fewer reagents are needed and sample preparation is faster.

In general, in the combinatorial detection schemes described herein, N may be 4, 5, 6, 7, 8, 9, 10, 11, 12, 13, 14, 15, 16, 17, 18, 19, 20, 21, 22, 23, 24, or higher, and K may be 2, 3, 4, or more, although in some embodiments, if K>N/2, the number of detectable species M may be reduced.

As discussed above, the methods described herein include one or more steps involving enzymatic deposition of oligonucleotide sequences (e.g., substrate-oligos) to the sample. In some embodiments, enzyme reporter molecules that include an oligonucleotide sequence conjugated to horseradish peroxidase (HRP) are used. The oligonucleotide sequence contains a region that recognizes and hybridizes specifically to the probe ID sequence in the probe assembly. This localizes HRP to the vicinity of an RNA analyte. A substrate-oligo molecule that includes an oligonucleotide sequence conjugated to a substrate such as tyramine is then introduced, and the HRP catalyzes deposition of substrate-oligo molecules to bind covalently to the sample.

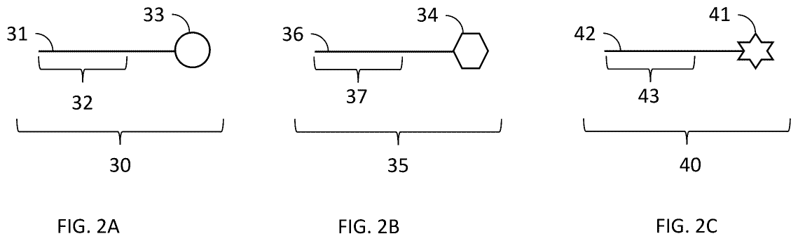

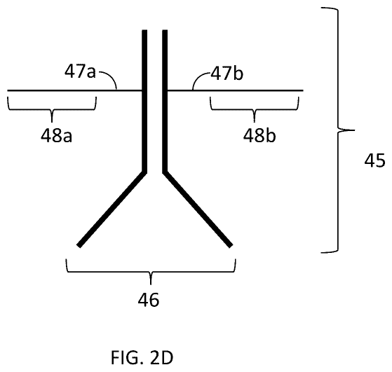

FIG. 2A is a schematic diagram showing an example of an enzyme reporter molecule 30. The enzyme reporter molecule includes an oligonucleotide sequence 31 conjugated to enzyme 33. Sequence 31 includes a probe ID recognition sequence 32.

While HRP is used as the enzyme in some embodiments, other enzymes and catalytic and/or reactive species can be used instead according to experimental preferences and the situation at hand. Alternatives such as artificial enzymes or alkaline phosphatase can be used, if the corresponding substrate can be sufficiently activated through reaction to be effective at binding substrate-oligos to the sample.

Enzyme reporter molecules are commercially available from Advanced Cell Diagnostics for use with RNAscope Multiplex V2 assay. Oligonucleotides conjugated to HRP that are suitable for use as enzyme reporter molecules are also available from Integrated DNA Technology (Coralville, IA).

FIG. 2B is a schematic diagram showing an example of a substrate-oligo molecule 35. The substrate-oligo molecule includes an oligonucleotide sequence 36 with a barcode ID sequence region 37 conjugated at either the 5′-end or the 3′-end to a suitable substrate 34 for the HRP enzyme. Suitable substrate molecules for the HRP enzyme include those that have been previously reported as substrates that can act as part of an amplified reporter system. The amplified reporter process involves the enzyme-mediated deposition of phenol-containing “detectable” molecules to adjacent molecules in the vicinity of the localized HRP enzyme. This amplified deposition process works as follows: in the presence of hydrogen peroxide, a localized HRP enzyme activates phenolic molecules to phenoxyl radicals that can then covalently bind to protein side chains, such as tyrosine side chains, leading to deposition of a molecule that can be detected by virtue of the detectable group. In the present disclosure, the detectable group is the oligonucleotide sequence.

Suitable phenol-containing substrate molecules include, but are not limited to, tyramine, tyramide derivatives, and the molecules described in U.S. Pat. No. 5,863,748, the entire contents of which are incorporated herein by reference. Suitable substrate molecules can also include either trans or cis isomers of p-hydroxy-cinnamic acid, or any derivative of p-hydroxy-cinnamic acid. Additional suitable derivatives of p-hydroxy-cinnamic acid include, but are not limited to, derivatives described in Taofiq, et al., Molecules, 22 (2):281 (2017), the entire contents of which are incorporated by reference. These include, but are not limited to, sinapic acid, chlorogenic acid, rosmarinic acid, coumaric acid, caffeic acid, and ferulic acid, and their derivatives. Further suitable substrate molecules include, but are not limited to, any phenol molecule or phenol-derivative molecule that can act as a substrate for the HRP enzyme, such as those described in Colosi, et al., J. Am. Chem. Soc., 2006, 128, 4041-4047, the entire contents of which are incorporated herein by reference. These include, but are not limited to, phenol, 1,4-benzene-diol, 1,2,3-benzenetriol, 4-chlorophenol, 4-nitrophenol, 4-methoxyphenol, 4-ethylphenol, 4-ethoxyphenol, 2,6-dimethoxyphenol, 4-tert-butylphenol, 4-phenylphenol, bisphenol A, 4-octylphenol, 17-beta-estradiol, and 17-alpha-ethynylestradiol, and their derivatives.

Additional suitable phenolic derivatives that have been shown to function as part of an amplified analyte detection methodology are described in U.S. Patent Application Publication No. US 2020/0378975, the entire contents of which are incorporated herein by reference. The phenolic derivatives therein specifically rely on the conjugation of the phenolic derivative to detectable groups that are not contained within the group of molecules of oligonucleotides or oligonucleotide sequences. Thus, the present disclosure includes, but is not limited to, oligonucleotide conjugates of the styryl phenols and derivatives contained within U.S. Patent Application Publication No. US 2020/0378975.

Embodiments of the methods described herein can utilize only one substrate-oligo molecule or a plurality of different substrate-oligo molecules. When more than one substrate-oligo molecule is employed, the barcode ID sequences are preferably orthogonal, meaning reporter molecules that recognize a particular barcode ID sequence have low cross-reactivity with other barcode ID sequences used. The design and verification of sequence cross-reactivity is known to those skilled in the art of DNA probe design.

The substrate-oligo species can be deposited as single-stranded DNA (ssDNA) or as double-stranded DNA (dsDNA) where an oligonucleotide sequence that includes the barcode ID recognition sequence, or a subset thereof, is hybridized to the barcode ID sequence or a portion thereof.

The length and composition of sequence 36 may be chosen according to the needs at hand. The barcode ID sequence 37 is used to selectively localize reporter molecules that recognize the barcode ID sequence and hybridize to it. The binding affinity should generally be sufficient to keep the reporter molecules localized during detection.

FIG. 2C is a schematic diagram showing an example of a reporter molecule that can be used in the methods described herein. Reporter molecule 40 includes a dye molecule 41 conjugated to an oligonucleotide sequence 42 that includes a barcode recognition sequence region 43. The barcode recognition region recognizes and binds to the barcode ID sequence of the deposited substrate-oligos as described above.

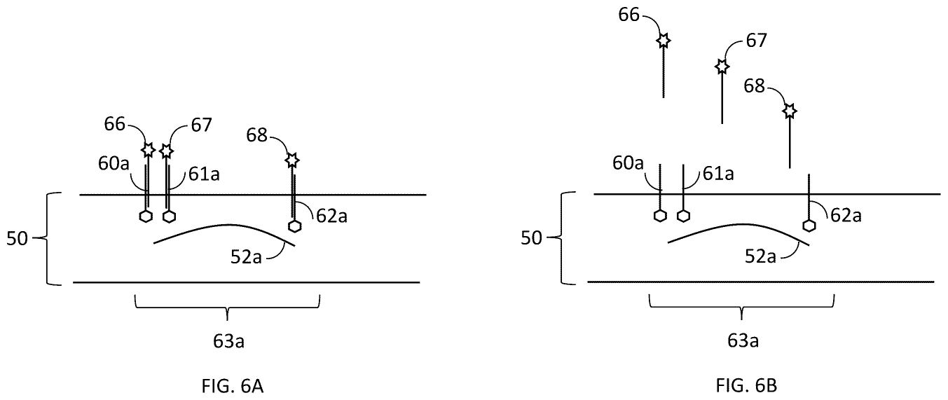

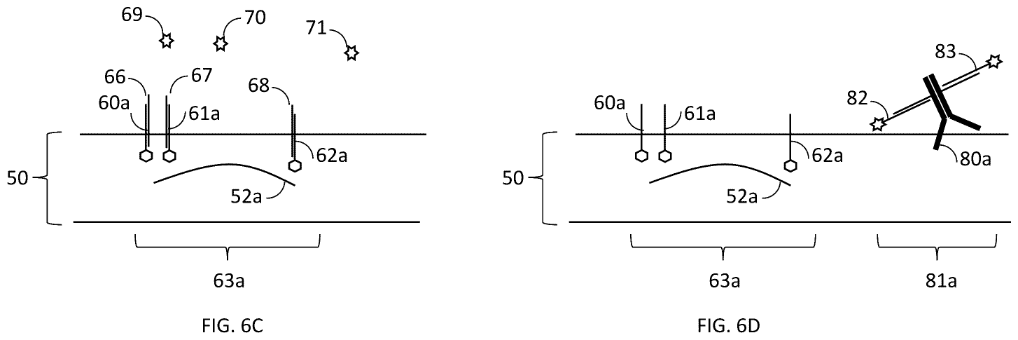

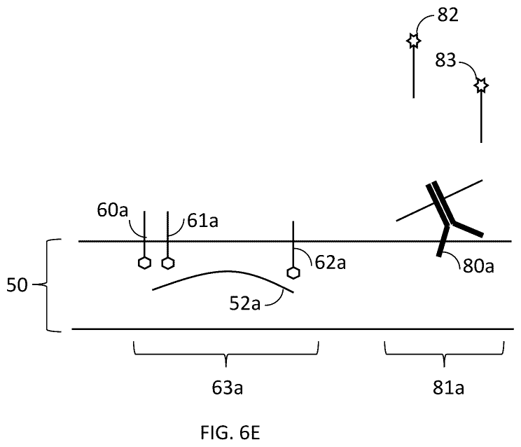

FIG. 6A is a schematic diagram showing hybridization of reporter molecules to substrate-oligos in a sample for imaging and detection of target RNA analytes. Substrate-oligos 60a-62a have been deposited in sample 50 in region 63a, localized near target analyte 52a. Reporter molecules 66-68 hybridize to the barcode ID sequence regions of the substrate-oligos and can generate signals representative of the presence of analyte 52a at location 63a in the sample.

In some embodiments, after the reporter molecules in the sample have been imaged, the reporter molecules are dehybridized to remove the dye from the sample and enable subsequent imaging cycles without the dye signal present. In these embodiments, dehybridization may be accomplished by elevating the temperature; denaturing with a suitable chemical agent such as DMSO, formamide, urea, guanidium chloride; using a toehold release strategy; a single-strand binding agent; or hybridizing an oligonucleotide with greater affinity such as PNA. The Tₘ of the hybridization interaction is chosen to ensure selective localization during imaging conditions while enabling removal of reporter molecules by the chosen method. Preferably Tₘ for the pair comprising the barcode ID sequence and the reporter molecule barcode ID recognition sequence is between 35° C. and 45° C., or between 35° C. and 50° C., or between 35° C. and 60° C., or between 35° C. and 65° C., or between 35° C. and 70° C., or between 35° C. and 75° C.

FIG. 6B is a schematic diagram showing the sample of FIG. 6A. After imaging, reporter molecules 66-68 are dehybridized from substrate-oligos 60a-62a and are removed from sample 50, e.g., by washing.

Suitable examples of dye molecule 41 include, but are not limited to, fluorescent labels include xanthene dyes, e.g., fluorescein and rhodamine dyes, such as fluorescein isothiocyanate (FITC), 6-carboxyfluorescein (commonly known by the abbreviations FAM and F), 6-carboxy-2′, 4′, 7′,4,7-hexachlorofluorescein (HEX), 6-carboxy-4′,5′-dichloro-2′, 7′-dimethoxyfluorescein (JOE or J), N,N,N′,N′-tetramethyl-6-carboxyrhodamine (TAMRA or T), 6-carboxy-X-rhodamine (ROX or R), 5-carboxyrhodamine-6G (R6G⁵ or G⁵), 6-carboxyrhodamine-6G (R6G⁶ or G⁶), and rhodamine 110; cyanine dyes, e.g., Cy3, Cy5 and Cy7 dyes; coumarins, e.g., umbelliferone; phenanthridine dyes, e.g., Texas Red; ethidium dyes; acridine dyes; carbazole dyes; phenoxazine dyes; porphyrin dyes; polymethine dyes, e.g., BODIPY dyes and quinoline dyes; Atto dyes (ATTO-TEC GmbH, Siegen Germany); DyLight dyes (Dyomics GmbH, Jena, Germany); AlexaFluor dyes (ThermoFisher Scientific); and CF dyes (Biotium, Fremont CA).

In some embodiments, multiple different reporter molecules are introduced into the sample in a single imaging cycle. When this occurs, the dye molecules of the different reporter molecules can be optically distinct from one another. Examples of distinct groupings of dyes that can be imaged with little interaction include AlexaFluor 488, Atto 550, AlexaFluor 647, and AlexaFluor 750; FITC, Cy3, Cy5, and Cy7; and CF488, 555, 647, and 750. Further, integrated DNA Technologies (Coralville, IA) supplies commercially available oligonucleotide sequences conjugated to AlexaFluor dyes that are suitable for the methods herein.

In some embodiments, the reporter molecule is introduced to the sample; localizes at barcode IDs based on hybridization of the barcode recognition sequence 43 to a selected barcode ID sequence; a sample image is acquired; and the reporter molecue is dehybridized from the barcode ID sequence for removal (e.g., by washing) from the sample.

In certain embodiments, the reporter molecule is not dehybridized; rather, the dye molecule 41 is cleaved from the nucleotide sequence 42. For this, a variety of chemical-based or photo-induced cleavage methods may be used. In some embodiments, the reporter molecule may contain a chemically or photo-cleavable linkage so it can be fragmented by exposure to a chemical or to light. For example, the dye molecule may be conjugated to the nucleotide sequence 42 via a disulfide bond and cleaved after imaging by exposure to tris (2-carboxyethyl) phosphine (TCEP), β-mercaptoethanol or other reducing agents. Other chemically cleavable bonds will be apparent to those skilled in the art or are described, for example, in Brown, Contemporary Organic Synthesis 4 (3):216-237 (1997).

In some embodiments, the dye molecule may be linked to the nucleotide sequence 42 using a UV photocleavable (“PC”) linker such as ortho-nitrobenzyl-based linkers or the dye can be inactivated in-place by photobleaching or chemically altering the dye. Use of cleavable bonds is suitable provided the cleaving method does not damage the substrate-oligos or labeled antibodies.

FIG. 6C shows an example of a sample 50 that includes a target RNA sequence 52a. Substrate-oligos 60a-62a have been durably bound to the sample in region 63a, and reporter molecules 66-68 have hybridized to the substrate-oligos. Following imaging of the reporter molecules in the sample, dye molecules 69-71 are cleaved from the oligonucleotide sequences of the reporter molecules, which remain hybridized to the respective substrate-oligos.

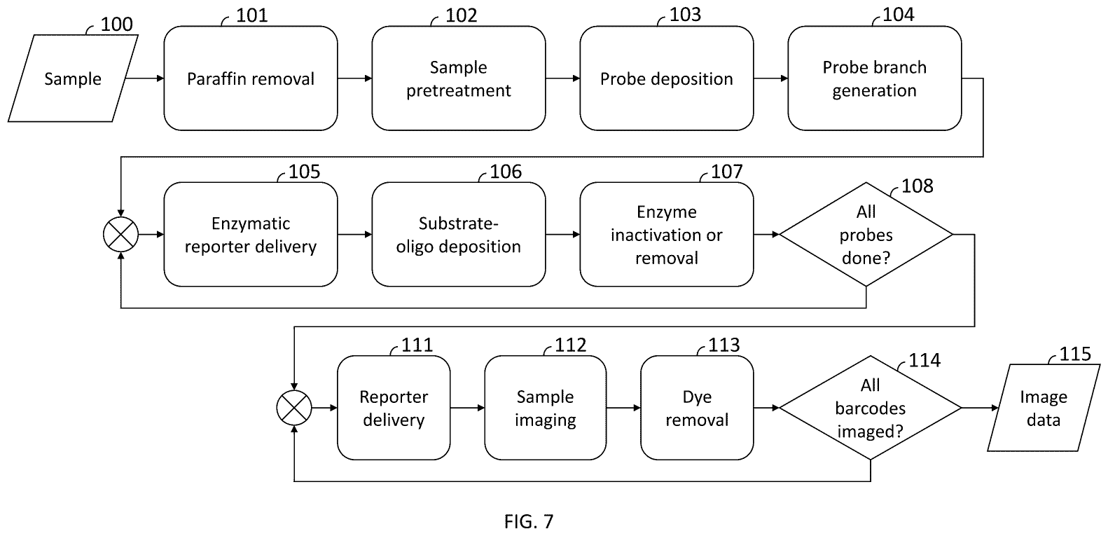

The overall method is depicted in the flow chart of FIG. 7, which shows a series of example steps for detecting RNA target analytes in a sample. The sample 100 can be a section (e.g., a 4 micron-thick section) cut from a formalin-fixed, paraffin-embedded (FFPE) lung cancer biopsy sample from a human patient. Other exemplary samples of interest include, without limitation, both tumor and non-neoplastic biopsies of skin, soft tissue, bone, breast, colon, liver, kidney, adrenal, gastrointestinal, pancreatic, gall bladder, salivary gland, cervical, ovary, uterus, testis, prostate, lung, thymus, thyroid, parathyroid, pituitary, brain, spinal cord, ocular, nerve, and skeletal muscle.

Optional paraffin removal step 101 can be performed in a variety of ways, including baking followed by progressive washes in xylene, EtOH, and water. Alternative protocols substitute less-toxic solvents for xylene. These are known to those skilled in the art, and the decision to use one protocol or another can depend on the type of paraffin used to embed the sample. In some embodiments, paraffin removal step 101 is performed by baking for 1-2 hours in a 60° C. incubator, followed by three 5-minute baths of Histoclear™ (available from Electron Microscopy Sciences, Hatfield, PA) and gradient hydration via successive 5-minute baths of 100% EtOH, 100% EtOH, 90% EtOH, 70% EtOH, and double-distilled water (ddH₂O), all at room temperature.

In certain embodiments, the sample 100 is not an FFPE tissue section. Instead, without limitation, it may be a fresh frozen section; cell pellet; a monolayer of cells such obtained as a fine-needle aspirate (FNA) or cytospin preparation; for these, there is no need to perform paraffin removal in step 101. Instead, the sample may be subjected to fixation with formalin, PFA, ice-cold MeOH, or acetone, as is known in the art of histology.

Sample pretreatment 102 has as its goal to render the RNA target molecules accessible to RNA probes, through permeabilization and other treatments. In this step and generally in the steps prior to imaging, materials should be RNAse-free. Pretreatment can include an antigen retrieval step. It may be protease-free, or it can include use of one or more proteases to partially digest proteins. It may include use of nonionic surfactants to promote permeabilization. It can optionally include a hydrogen peroxide treatment, to block or inactivate endogenous peroxidases.

One pretreatment protocol useful for the methods described herein begins with 3 successive baths in 1×PBS for 5 minutes each, after which the sample is put in a hot ddH₂O steamer for 10 seconds. It then undergoes antigen retrieval at 90-95° C. for 10 minutes, after which it is given two baths in ddH₂O for 1 minute each at the same temperature, and then one in 1×PBS at 40° C. The sample then undergoes protease digestion at 40° C. for 20 minutes, after which it is given 4 successive baths of 1×PBS for 1 minute each, at room temperature. Pretreatment concludes with room temperature fixation in 4% paraformaldehyde (PFA) for 5 minutes temperature followed by 3 one-minute baths in 1×PBS.

Optionally, the sample is incubated in a 0.3% hydrogen peroxide solution for 10 minutes up-front to block endogenous peroxidases.

Another suitable pretreatment protocol involves incubating for 10 minutes at room temperature in hydrogen peroxide, then removing any excess hydrogen peroxide and washing repeatedly in successive ddH₂O baths. Then the sample is incubated in RNAscope™ 1× Target Retrieval Reagent (available from Advanced Cell Diagnostics, Newark, CA) for 15 minutes in a 99C steamer. After rinsing in water, washing in ethanol for 3 minutes, and drying the sample, it is covered with RNAscope™ Protease Plus Reagent (available from Advanced Cell Diagnostics, Newark, CA) and incubated from 15 to 30 minutes at 40C. Finally, it is washed twice in fresh ddH₂O.

During probe deposition step 103, RNA probes are incubated with the sample and hybridize with target sequences of the RNA analytes being detected. Time, temperature, concentration, and the buffer composition (surfactant, stringency) can be optimized for a given probe type. The probe manufacturer's recommended protocol can be used and adjusted only if results show that is necessary or yields improved results.

For example, a sample can be prepared using a View RNA™ assay probe kit (available from ThermoFisher Scientific, Waltham, MA) targeting PPKB, POLR2A, UBC, and LDHA. For this assay, probe deposition 103 generally follows the vendor's recommendations. The probes are applied and sit for 2 hours at 40° C. to hybridize with RNA in the sample, then are subjected to 3baths in View RNA™ Wash Buffer for 2 minutes with frequent agitation.

In some embodiments, probe assemblies do not involved branch generation and step 104, which is optional, can be omitted. For example, when using probes of the type shown in FIGS. 1A and 1B, the enzyme reporter molecules recognize probe ID sequence 13 or the combined sequences 18a and 18b, respectively.

Other types of probe assemblies create a structure of additional molecules that provide the probe ID sequences recognized by the enzyme reporter molecules. For such probes, branch generation step 104 is performed according to the manufacturer's recommendations for the probe being used. Probes that use a preamplifier and amplifier scheme typically employ a hybridization step for that element to bind to the combined sequence 18a and 18b, followed by washes, and then a hybridization step to attach the amplifier molecules to the preamplifier molecules, followed by more washes.

For example, a sample prepared with 4 View RNA™ probes is incubated for 30 minutes in View RNA™ PreAmp mix in a hybridization oven at 40° C., washed three times in View RNA™ Wash Buffer for 2 minutes each with frequent agitation. It is then incubated for 30 minutes in View RNA™ Amplifier mix at 40° C. and washed three times in View RNA™ Wash Buffer for 2 minutes each with frequent agitation. This creates probe assemblies suitable for the methods described herein, targeting four specified RNA target analytes, with unique probe ID sequences for each probe assembly type. Other commercial probe kits provide protocols for generating branch structures atop the probes, and these are suitable for the methods disclosed herein.

Enzymatic reporter delivery step 105, substrate-oligo deposition step 106, and enzyme inactivation or removal 107 step are performed for one type of enzymatic reporter molecule at a time, and the sequence is repeated for each different enzyme reporter molecule until all probe ID sequences have been processed. Decision step 108 regulates the number of cycles that are performed.

To deposit enzyme reporter molecules in the sample, in some embodiments, sufficient reporter molecules are applied to cover the sample, which is then incubated at 40° C. for 20 minutes then washed three times in View RNA™ Wash Buffer at room temperature to remove excess enzyme reporter molecules from the sample.

In certain embodiments based on RNAscope™ v2 probes, there are three species of enzyme reporter molecules, denoted RNAscope™ Multiplex FL v2 HRP-C1, RNAscope™ Multiplex FL v2 HRP-C2, and RNAscope™ Multiplex FL v2 HRP-C3 (all available from Advanced Cell Diagnostics, Newark, CA). Step 105 is performed by covering the sample with the enzyme reporter molecules and incubating at 40° C. for 15 minutes, then washing at room temperature in two successive baths of 1×wash buffer, for two minutes each.

Substrate-oligo deposition 106 step involves incubating the sample in substrate-oligo diluted in TSA buffer (available from Akoya Biosciences, Menlo Park, CA). Incubation time affects the amount of amplification and best times can be found by experimentation. Typically, best results are obtained for incubation times between 2 minutes and 30 minutes. In some embodiments, p-iodo-phenylboronic acid (PIBA) is mixed with substrate-oligo in a 20:1 (w/w) ratio prior to dilution in TSA signal amplification buffer, and then incubated for 20 minutes at 40 C. The use of PIBA and other compounds in connection with HRP-catalyzed deposition is described in U.S. Pat. No. 6,593,100, the entire contents of which are incorporated by reference herein. After incubation, the sample is washed three times in PBS for 2 minutes each with frequent agitation.

In this step, TSA-plus signal amplification diluent can be substituted for TSA signal amplification diluent to further enhance signal.

In some embodiments, the substrate-oligo molecule includes an oligonucleotide sequence with an 18-mer barcode conjugated to hydroxycinnamic acid (HCA). The synthetic procedure for the oligonucleotide-substrate conjugate involved the coupling of trans-4-hydroxy-cinnamic acid (p-coumaric acid) to an amine-modified oligonucleotide following EDC/sulfo-NHS coupling conditions. This conjugation method is a standard amide-bond method of conjugation that is well-known to those skilled in the art (see, e.g., Bartczak & Kanaras, Langmuir 2011, 27, 10119-10123, the entire contents of which are incorporated by reference herein). Analysis of the purified oligonucleotide conjugate by reverse-phase HPLC demonstrated one major peak, approximately 75% pure, in the chromatogram (Agilent Zorbax 300SB-C8 column with triethylammonium acetate pH 7.0/acetonitrile as the mobile phase). The purified oligonucleotide was dissolved in nuclease-free water at a concentration of approximately 485 uM and used without further purification in the assays. This process can be performed for each of P distinct barcode ID sequences to produce P substrate-oligo molecules for use in the methods described herein.

Alternative methods for the synthesis of various sequences of oligonucleotide-substrate conjugates can also be used. A common and efficient method for preparing a large number of different oligonucleotide conjugates involves using solid-phase or pseudo-solid phase approaches as described by Franzini, et al. (Bioconjugate Chem. 2014, 25, 1453-1461), the entire contents of which are incorporated herein by reference. Other conjugation approaches involve copper-catalyzed alkyne-azide cycloaddition (click-chemistry), copper-free DIBCO/DIBO-azide conjugation, reductive amination, the use of heterobifunctional cross-linkers such as SANH or SMCC (Gong, et al., Bioconjugate Chem. 2016, 27, 1, 217-225, the entire contents of which are incorporated herein by reference), photochemical conjugation, aminooxy-ketone conjugation, Staudinger ligation, Diels-Alder cyclo-additions, thiol-maleimide conjugations, enzyme-catalyzed ligations, in addition to other oligonucleotide conjugation chemistry as described within Singh, et al., Current Org. Chem, 2008, 12, 263-290, the entire contents of which are incorporated herein by reference.

It is often a goal in RNA target detection to produce fine punctate spots corresponding to individual RNA molecules in the sample. Also, the number of sites for enzymatic deposition of substrate-oligos can be limited in some samples. For either or both these reasons, it can be valuable to adjust the degree of enzymatic amplification so that the spots are not overly large, nor does deposition of substrate-oligos for a given RNA analyte so deplete the surrounding region of binding sites that substrate-oligos in subsequent deposition cycles are unable to bind.

This consideration is also important when each RNA analyte is targeted by multiple probes to perform combinatorial or confirmational RNA analyte detection. If the enzymatic amplification is too high, later-deposited substrate-oligos will be more-weakly deposited than earlier substrate-oligos, leading to false negatives in the combinatorial or confirmational detection scheme.

As a result, excessive amplification can be a concern when using probe assemblies that can provide many enzymatic reporter molecules per probe assembly. Conversely, when using simple probes and probe assemblies like those in FIGS. 1A and 1B with a single probe ID sequence region per probe or probe assembly, a high degree of enzymatic amplification may be typically desirable since there is at most one enzyme reporter molecule per probe.

Amplification can be adjusted upward by increasing concentration of the substrate-oligos, longer incubation time, elevated temperature, and the use of p-iodo-phenylboronic acid, PIBA, or other compounds in the buffer used for deposition. Reversing these will reduce amplification. During assay development, the amplification can be adjusted as necessary to balance concerns about puncta size, binding site depletion, and signal level.

Enzyme inactivation or removal step 107 has as its goal the removal or inactivation of the enzyme in the enzyme reporter molecules. For HRP-containing enzyme reporter molecules, this can be done using known methods for inactivating HRP. Examples include incubation for 15 minutes at 40° C. with RNAscope™ Multiplex FL v2 HRP blocker (available from Advanced Cell Diagnostics, Newark, CA); or incubation for 10 minutes in BLOXALL™ (available from Vector Labs, Newark, CA); or incubation for 15 minutes at room temperature in ReadyProbes™ Endogenous HRP and AP blocking solution (available from ThermoFisher Scientific, Waltham, MA); or incubation for 15 minutes at room temperature in a 3% hydrogen peroxide/MeOH solution. The choice of one inactivation approach over another and adjustment of incubation conditions can be done by assessing carry-over signal from one enzyme reporter (i.e., RNA dot pattern) into the signal of the subsequent reporter and its RNA pattern, when the sample is imaged with the reporter molecules.

If the affinity of the enzymatic reporter molecule to the probe ID sequence is sufficiently low compared with that of the probe binding to the RNA and of any hybridized molecular structures such as preamplifier molecules and amplifier molecules, it is possible to remove the enzyme reporter molecules without disrupting the remaining probes bound to the sample. This can be accomplished by use of elevated temperature, or denaturing buffers such as 60% DMSO, 70% DMSO, 80% DMSO, or 90% DMSO, or both. However, the Tₘ of the probe recognition sequence pairing should typically be well above Tₘ of the probe ID sequence pairing, with a difference of at least 10° C., or 15° C., or at least 20° C., or at least 25° C. or even more. Otherwise, it is difficult to produce conditions that remove the enzyme reporter molecules without at least partially degrading the RNA probe or probe assembly binding.

Steps 105 through 107 are repeated for each combination of enzymatic reporter molecule and substrate-oligo in turn. At that point, the sample has substrate-oligos with a first barcode ID sequence durably bound to the sample at locations corresponding to the first RNA probe; substrate-oligos with a second barcode ID sequence durably bound at locations corresponding to the second RNA probe, and so on, for all sets of RNA probes and substrate-oligos with barcode

ID sequences. In general, the number of different barcode ID sequences among the substrate-oligos that are deposited in the sample can be 1 or more, e.g., 2 or more, 3 or more, 4 or more, 5or more, 7 or more, 10 or more, 15 or more, 20 or more, or even more.

The preceding steps can be performed manually or using an autostainer, which makes it practical to perform many deposition cycles.

The process of detecting the RNA target analytes includes three repeated steps: reporter molecule delivery 111, sample imaging 112, and dye removal 113. Each time these steps are performed, a plurality D of reporter molecules can be imaged, based on the number of optically distinct dyes employed. If the total number of probe ID sequences N exceeds D, then multiple readout cycles are used to read all RNA species. In multiomics embodiments with labeled antibodies, the total number of images involved increases by the number of antibody species, increasing the number of readout cycles used.

This process can be automated using a fluidics chamber connected to a fluidics delivery system with the ability to introduce and remove reagents while the sample is on a microscope stage and can be imaged. The fluidics chamber can be an open-well arrangement like the PhenoCycler™ Open (available from Akoya Biosciences, Menlo Park, CA), or a flow-cell arrangement like the Phenocycler™ Fusion (available from Akoya Biosciences, Menlo Park, CA).

An automated system can be assembled by coupling fluidics apparatus to a flow-cell as is described in U.S. Patent Application Publication No. 2020/00393343, the entire contents of which are incorporated by reference herein, together with a microscope and control-electronics to oversee its operation. The functional aspects for automation is that the system is able to position the sample so it can be imaged, and that it can deliver and remove reagents to and from the sample surface. The reagents include the reporter molecules and a counterstain such as DAPI or Hoechst, along with buffers and denaturing or cleaving compounds as will be described below.

Alternatively, imaging can be done manually, meaning the sample is iteratively imaged and moved off-instrument for fluidic processing. The sample can be located on a coverslip and imaged with an inverted microscope, or on a slide to which a coverslip is temporarily attached for imaging and then removed for subsequent fluidic processing. This cycle is repeated to perform the overall experiment.

In a preferred embodiment, when step 111 is performed the first time, DAPI is delivered with no accompanying reporter molecules. After a 3-minute incubation, the sample is washed with a solution of 20% DMSO and 80% saline buffer, and the sample is imaged using filters selected for detecting DAPI and the reporter dyes.

In one example embodiment, the reporter molecule dyes are FITC, Atto 550, Cy5, and AlexaFluor 750 and the microscope contains a triple-band epi-filter for imaging DAPI, Atto 550, and AlexaFluor 750; and a double-band epi-filter for imaging FITC and Cy5. An agile LED illuminator excites a single band at a time, and an automated filter changer selects the desired epi-filter so all 5 bands are obtained under computer control.

The resulting image obtained in step 112 indicates the signal levels in each band for all points in a region of the sample. The DAPI signal can be used for sample location and focusing. Signal in the non-DAPI channels indicates background emission due to sample auto-fluorescence and similar phenomena. This signal can be high in FFPE tissue, especially in the shorter-wavelength bands such as Atto 550 and below. An image of this type, with no reporters present, is herein termed a background image.

In step 113, the dye molecules are removed. The dye molecules can be removed by dehybridizing the reporter molecules, using successive washes of 90% DMSO/10% saline buffer, 20% DMSO/80% saline buffer, and pure saline buffer. The first wash lasts 5 minutes, and the others last 2 minutes each; the sequence of three washes is repeated twice.

Steps 111 through 113 are repeated until all reporter molecules have been imaged, as denoted in decision step 114. The image data is then optionally further processed in step 115.

In some embodiments, in subsequent cycles, reporter molecules are introduced in step 111, in a 20% DMSO/80% buffer solution. They localize at substrate-oligo molecules having the barcode ID sequence associated with their barcode recognition sequence, hence at sites corresponding to RNA analytes targeted by probes with the associated probe ID sequence. After incubation and hybridization, the sample is washed with 80% buffer. Alternative buffers can be used to deliver the reporters, such as saline sodium citrate, SSC, or other buffers. This may be done to promote hybridization or adjust stringency of reporter molecule binding.

In an example embodiment, four reporter molecules are introduced, each with a unique barcode ID recognition sequence, and each reporter molecule in an imaging cycle contains a different dye. One uses FITC, the second uses Atto 550, the third uses AlexaFluorCy5, and the fourth uses AlexaFluor 750, for example.

The sample is imaged again in step 112. The resulting signals indicate the combined effect of the reporter molecules, localized to the sites of the substrate-oligos that are bound to the sample, plus background emission. An image of this type, including reporter molecule signal plus background, is herein termed a raw signal image.

The dye removal step 113 is repeated. While dehybridization of the reporter molecule can be used, alternatives such as removing the dye by chemical cleaving, photo-cleaving, photobleaching, or chemical inactivation of the dye can also be used. The important aspect is that there is generally little or no dye signal from the reporter molecules in subsequent imaging cycles.

This process continues until all images have been obtained. At this point, one more cycle of steps 111-113 can optionally be performed, supplying only the counterstain with no reporter molecules. The image obtained in step 112 should have no reporter molecule signal contribution, so will be a background image similar to the one optionally acquired prior to any reporter molecules being applied. However, it may differ because the sample autofluorescence properties were slightly altered by the intervening imaging and chemical processing steps. Also, in some embodiments, no initial background image is acquired. Overall, there is typically at least one background image in order to correct for sample background fluorescence, and it can be valuable to have more than one in order to measure and compensate for changes in sample properties during the experiment. In some embodiments, additional background images are acquired between successive reporter molecule deposition cycles, by omitting all reporter molecules. This can be desirable if the total number of cycles is very high, and the sample is a type whose background fluorescence is observed to vary greatly during the experiment.

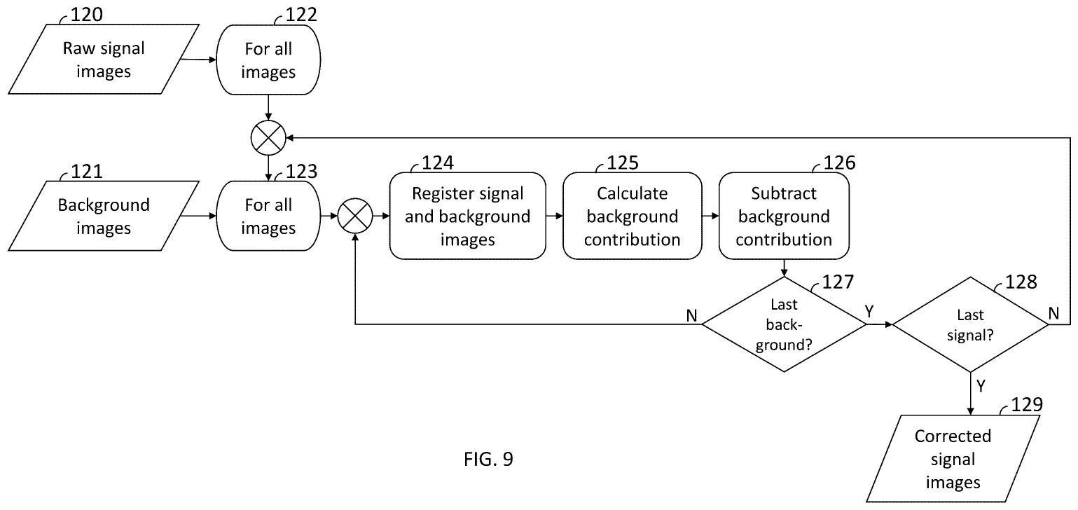

Once all the images are collected, optional background correction can be performed, one method for which is illustrated in the flow chart of FIG. 9. In steps 120, 121, 122, and 123, raw signal images and background images are obtained for the sample in the manner described above.

Next, in step 124, each raw signal image is registered to the background image(s). Step 125 indicates calculating the contribution from each background image to subtract from the raw signal image. For example, if there is only one background image, the contribution would be 100% of the background image from all raw images. But if there are multiple background images, the contribution can be based on which cycle the raw image was acquired in, to correct for sample changes during imaging. For example, in an experiment with a first and last background image and 3 cycles of reporter molecules, the weights might be proportionally assigned by cycle, as shown in Table 1:

TABLE 1

| Reporter | Initial | Final |
|---|---|---|
| molecule | background | background |
| cycle | weight | weight |
| 1 | 75% | 25% |
| 2 | 50% | 50% |
| 3 | 25% | 75% |

Once the weights are determined in step 125, the background contribution can be calculated for each pixel in the image, and the signals subtracted pixel-wise from the raw image to yield a corrected image saved among the results 129. These are corrected for tissue autofluorescence that otherwise can complicate visual review or reduce the sensitivity of downstream data analysis. The process repeats as shown by decision steps 127 and 128 until all desired signal images have been corrected.

The methods described herein can use combinatorial detection schemes for RNA analytes, where each RNA analyte is targeted with multiple probes, and the multiple probes include K different probe ID sequences. In this way, that RNA analyte is associated with K different enzyme reporters, substrate-oligos, and reporter molecules. The probe ID sequence assignments are chosen so that each RNA analyte has a unique combination of K reporter molecules, drawn from a total pool of N.

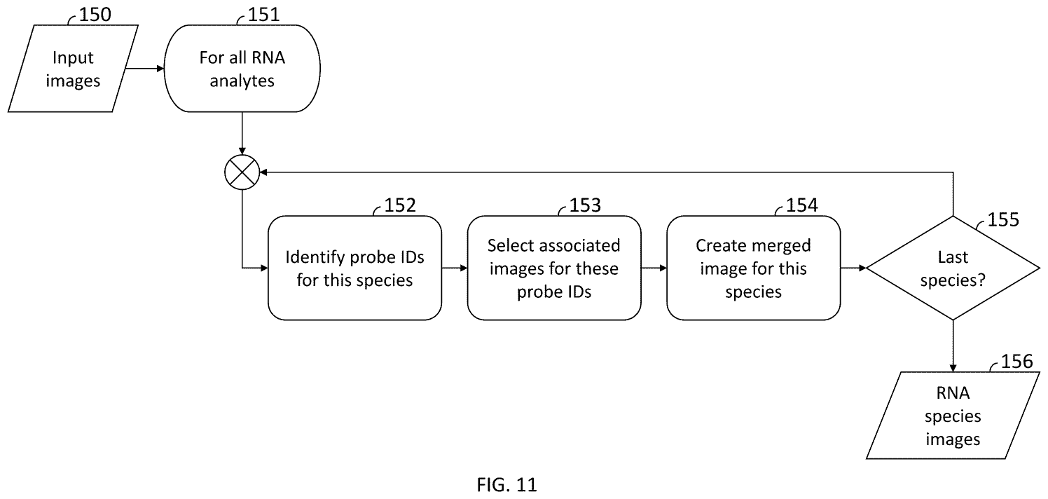

The resulting images can be interpreted to determine the presence and identity of RNA at every point in the image. An example sets of steps for implementing a combinatorial detection scheme is shown in FIG. 11. The steps correspond to a direct pixel-combination approach.

Images of the sample that include signals arising from the various reporter molecules are obtained in step 150 as described above; the reporter molecule signals correspond to each of the RNA analytes as shown in step 151, but do not correspond 1:1 to the RNA analytes.

Then, for each RNA analyte indicated in step 151, the method identifies the associated probe ID sequences in step 152 and hence, reporter molecules in step 153, then creates a merged image 154 that indicates presence of signal in all requisite reporter molecule images. This is repeated for all species combinations as shown in step 155 to create an RNA species image in step 156.

The merged image can use a pixel-value product, or a gating function whereby only signals above a threshold level contribute to the product, or other nonlinear terms to accommodate background or clip unexpectedly bright signals in a given reporter molecule image. It may include a spatial filter on each image contributing to the merged image.

Other algorithms are possible, such as making a provisional dot-detection for each input image, and using proximity and identity of provisional dots in each reporter channel to call the dot as positive for a selected RNA species, or negative if the combination is incomplete or ambiguous (i.e. matches multiple RNA signal combinations). Any method that performs the combination-assignment may be used, and the choice can be made based on simplicity, computational burden, or other factors according to the situation at hand.

In some embodiments, confirmational detection of RNA analytes is implemented, where each analyte is targeted with 2 probes, which include 2 different probe ID sequences. Once again, this means each RNA analyte is associated with a plurality of reporter molecules: two. However, the probe ID sequence assignment is performed so that each RNA analyte has a unique probe ID sequence, plus a probe ID sequence that is shared among all RNA analytes. It takes two detections to report an RNA analyte: one in its unique reporter molecule image (associated with its unique probe ID sequence), and one in the shared reporter image (associated with its shared probe ID sequence). This provides a guard against false detection by individual off-target probe binding, leading to improved specificity.

However, this scheme may, in some embodiments, reduce detection sensitivity, since fewer probes develop a signal for a given probe ID. Successful implementation of this method typically involves sufficient amplification via the probe design and the enzyme amplification to produce an adequate signal for detection. Techniques for this include use of more probes per RNA analyte, use of amplified probes with more probe ID sequence regions per probe, increased enzymatic amplification, and/or a combination of these.

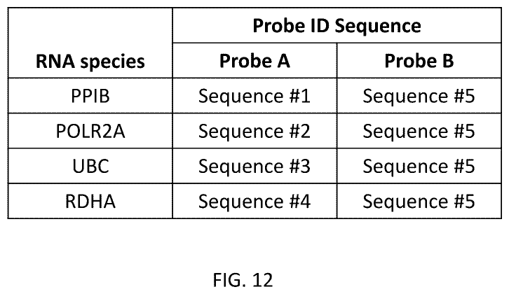

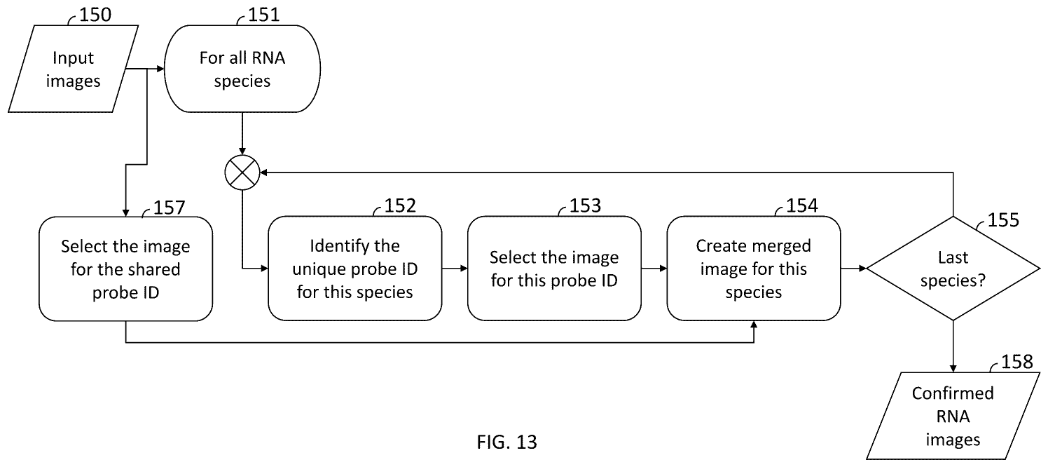

FIG. 12 shows example unique and shared probe ID sequences for the detection of 4 RNA target analytes using 5 sequences. Sequence #5 is shared among all RNA analytes, while Sequences #1-#4 are unique to only one of the RNA analytes. FIG. 13 shows an example set of steps for implementing a confirmational detection scheme, yielding confirmed RNA images from individual reporter images. Many of the steps of the method are similar to those of the method shown in FIG. 11. In step 157, the image corresponding to the shared probe ID sequence is identified and used in step 154 to create a merged image in step 154 for each RNA analyte. Based on the presence of signal arising from reporter molecules associated with both the analyte's unique probe ID and shared probe ID at locations in the sample, a confirmed RNA image is generated in step 158 that indicates the presence of the RNA analyte at sample locations.

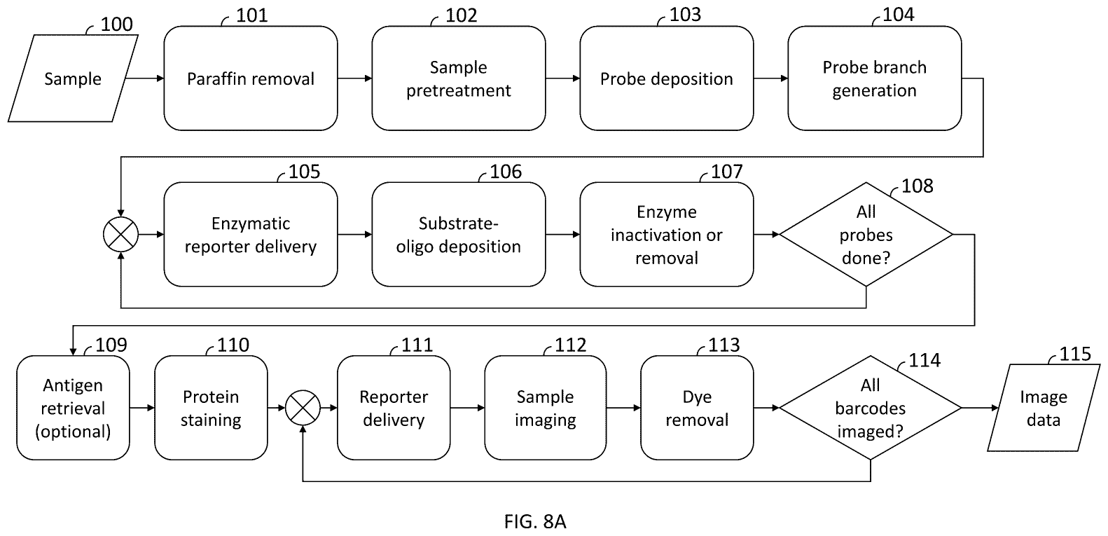

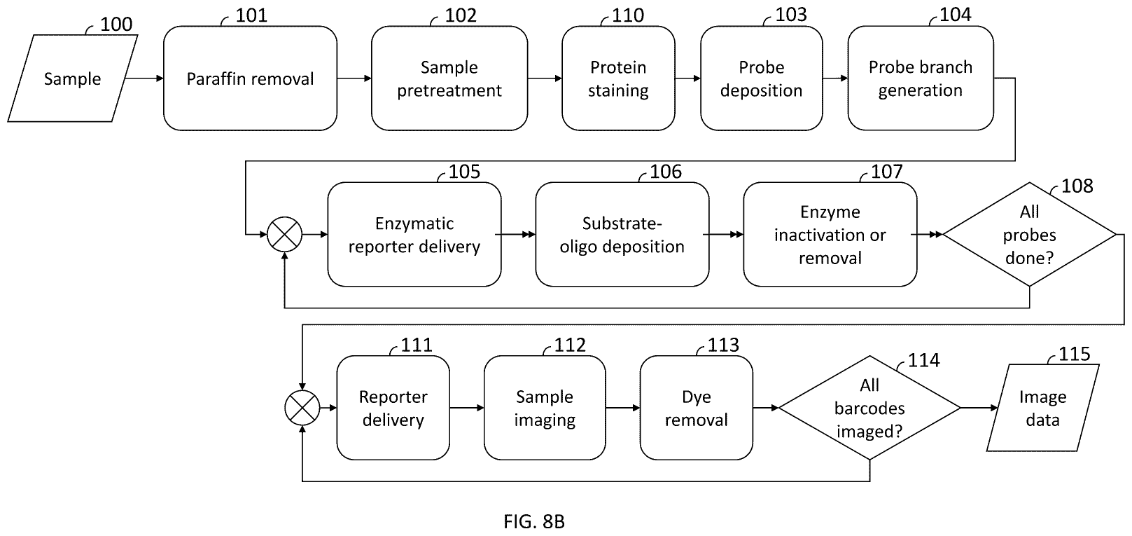

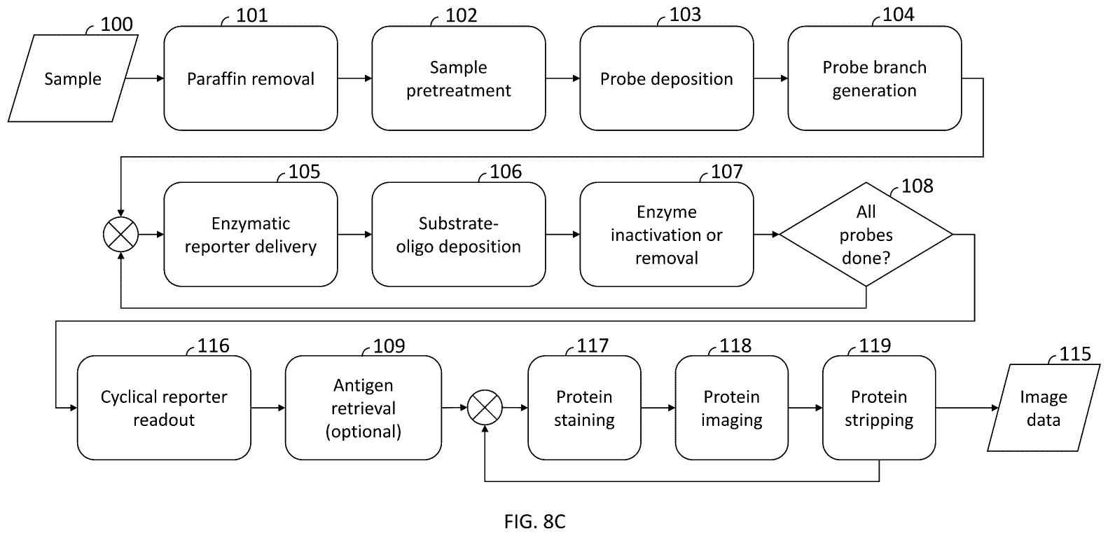

In some embodiments, the workflows described herein implement multiomic workflows for detection of RNA analytes and proteins peptides, and/or amino acid sequence targets in the sample. FIG. 8A shows an example set of steps for performing an RNA-first multiomics workflow, and FIG. 8B shows an example set of steps for performing a protein-first multiomics workflow.

For detection of proteins, peptides, and amino acid sequence targets, multiomics workflows can use of labeled affinity molecules as probes. An example of a probe for proteins, peptides, and/or amino acid target sequences using an antibody as the affinity molecule is depicted in shown in FIG. 2D. Probe 45 includes an antibody 46 labeled with one or more oligonucleotide sequences, each having a barcode ID region. In this example, two sequences are shown, depicted as 47a and 47b, with barcode ID regions 48a and 48b respectively. While two sequences are depicted, the number can be higher, or as low as 1. Also, the number can vary for individual antibody molecules. The sequence of the barcode ID region(s) is associated with the protein, peptide, or amino acid target. Reporter molecules that selectively bind to barcode ID sequences can be introduced into the sample and hybridize to the barcode ID sequences, and then be imaged as described above to obtain location information for target proteins, peptides, and/or amino acid sequences in the sample.

While an antibody is shown in FIG. 2D, it should be appreciated that the affinity molecules can be antibodies, or aptamers, or nanobodies, or any other molecule which exhibits specific affinity for the protein/peptide/amino acid target of interest. The oligonucleotide labels featuring barcode IDs can be connected to the affinity molecule by conjugation or complexation. Methods for conjugation are known in the art, and can involve amide-bond conjugation via activated carboxylic acids, copper-catalyzed alkyne-azide cycloaddition (click-chemistry), copper-free DIBCO/DIBO-azide conjugation, reductive amination, the use of heterobifunctional cross-linkers such as SANH or SMCC (Gong, et al., Bioconjugate Chem. 2016, 27, 1, 217-225, the entire contents of which are incorporated herein by reference), photochemical conjugation, aminooxy-ketone conjugation, Staudinger ligation, Diels-Alder cyclo-additions, thiol-maleimide conjugations, enzyme-catalyzed ligations, in addition to other oligonucleotide conjugation chemistry as described within Singh, et al., Current Org. Chem, 2008, 12, 263-290, the entire contents of which are incorporated herein by reference.

The oligonucleotide label may be connected directly to the affinity molecule, or it may be connected via hybridization with oligonucleotide linkers, trees, or other structures.

In some embodiments, the affinity molecules do not have oligonucleotide labels, and they are detected via direct dye conjugation to the affinity molecule, or via labeled secondary antibodies that recognize and bind to the affinity molecule. The readout scheme is different for multiomics experiments of this type, and they preferably implement the RNA-first workflow shown in FIG. 8A.

FIG. 5B is a schematic diagram showing a sample 50 that is subjected to a multiomics workflow. Following the partial or complete removal of probe assemblies 51a and 51b (as described previously), probes are introduced to specifically bind to protein, peptide, and/or amino acid target analytes in the sample. In FIG. 5B, probes 80a and 80b bind to analytes in regions 81a and 81b of sample 50, respectively. The probes can include antibodies as affinity molecules, or other types of affinity molecules or moieties as described herein.

FIG. 6D is a schematic diagram showing a portion of the sample of FIG. 5B after the introduction of reporter molecules. Specifically, reporter molecules 82 and 83 are introduced and hybridize to barcode ID sequence regions of oligonucleotide labels conjugated to the affinity molecules of probe 80a. The reporter molecules 82 and 83 generate optical signals (e.g., fluorescence) that can be imaged; locations of fluorescence emission indicate the location of the protein/peptide/amino acid sequence target analyte in sample 50 (i.e., in region 81a). Following imaging of the reporter molecules 82 and 83 in the sample, the reporter molecules are dehybridized from the oligonucleotide labels conjugated to the affinity molecules of probe 80a, and removed from the sample (e.g., by washing) as shown schematically in FIG. 6E.

Turning to FIG. 8A, many steps are the same as in the RNA-only workflow of FIG. 7. However, some steps may be performed differently for multiomic experiments involving both RNA and protein detection. In the sample pretreatment step 102, there is often a trade-off between optimizing for protein detection and optimizing for RNA detection. Use of longer or more aggressive antigen retrieval to enhance epitope availability for protein detection can damage or remove RNA species, and protease digestion steps that promote RNA detection can damage epitopes targeted by antibodies for protein detection. Protease-free sample pretreatment is typically preferred so long as the resulting RNA detection is sufficient.

In the workflow of FIG. 8A, protein staining and detection is not done until step 110, when RNA-associated detection is complete. At that point, substrate-oligos corresponding to the RNA species are durably bound to the sample and are unaffected by even aggressive antigen retrieval, if that is desired for the sake of protein detection.

Step 109 is thus a suitable point for optionally performing any antigen retrieval that involves pH or temperatures that would interfere or degrade RNA probes working successfully. The need for this depends on the sample types and epitopes of interest. In some cases, the antigen retrieval done as part of step 102 at an optimal level for RNA detection is sufficient for effective protein detection. In other cases, optimal protein detection requires a more aggressive antigen retrieval regimen or a different antigen retrieval regimen than that used for RNA detection, and it can be performed as part of step 109.

Antigen retrieval often involves use of temperatures of 60° C. or more, and often involves temperatures of 80° C. or higher. Such temperatures will dehybridize elements of many probes, such as removing the enzyme reporter molecules, or preamplifier molecules, or amplifier molecules, or removing probes from RNA entirely.

Step 109 may include measures to promote probe disassembly, regardless of whether antigen retrieval is performed or not. Probe disassembly can be desirable since the probes are no longer necessary and could they contain sequences that might cross-react with the protein-associated reporter molecules. Examples of such measures include use of denaturants, elevated temperatures, or both, in combination with wash steps.

Protein staining step 110 for labeled antibodies can be done following the protocol given in the CODEX™ User Manual (available from Akoya Biosciences, Menlo Park, CA at www.akoyabio.com/wp-content/uploads/2021/01/CODEX-User-Manual.pdf), the contents of which are incorporated herein by reference. This protocol includes DNA blocking with sheared salmon sperm and a mixture of counter-sense oligonucleotide sequences for all the barcode sequences in the labeled antibodies used. Staining can further incorporate use of a protein blocker such as serum or casein if that is beneficial, prior to antibody incubation. Antibody incubation is done with a cocktail of all antibodies, and can be done for 2 hours, or longer such as 4 hours, 6 hours, or 8 hours, or overnight. It can be done at reduced temperature such as 4° C. if desired.

After incubation, a cross-linking step is performed. This can be done with bis (sulfosuccinimidyl) suberate (BS3) as described in the CODEX™ User Manual, or with 4% paraformaldehyde (PFA), or other protein-crosslinking reagents.

Turning to the detection of reporter molecules, this can proceed in the same way as described for an RNA-only experiment, albeit with additional iterations of steps 111-113 to detect the protein-associated reporters. The RNA-associated images can be acquired first, or the protein-associated images, or they can be acquired in any order that suits the purpose at hand.

Other multiomic embodiments follow the protein-first workflow shown in FIG. 8B. For these, the protein staining step 110 is done prior to RNA probe deposition step 103. The antigen retrieval done during sample pretreatment step 102 generally balances any concerns of epitope availability against potential damage to RNA targets. Once again, the staining itself can be done using the protocol guidance given in the CODEX™ User Manual, with optional modifications to the blocking and fixation steps as noted.

After fixation, the protein affinity molecules remain on the sample through the RNA processing and are detected in the same way as described for FIG. 8A.

Other embodiments use protein affinity molecules without oligonucleotide labels. One example of this type of workflow is shown in FIG. 8C. In FIG. 8C, step 116 represents the combination of steps 111 and 114 in the previous figures, i.e. iteration over the RNA-associated reporter molecules. In this workflow, antigen retrieval step 109 may be aggressive, if desired, without affecting RNA detection just as in the workflow depicted in FIG. 8A. However, protein detection is done differently. Rather than staining with a composition of all antibodies that are detected via cyclic readout, primary antibodies are stained a few at a time in step 117. The antibodies are dye-conjugated, or further incubated with dye-labeled secondary antibodies or nanobodies that target species-specific sites on the primary antibodies. After washing steps, they are imaged in step 118. In such a scheme the number of primary antibodies present at any time is limited to the number of optical channels (or the number of species available if indirect readout is used). Thus only 2, or 3, or 4 antibodies are applied and imaged at a time. After imaging, they are stripped in step 119 and the cycle is repeated until all antibodies have been imaged. Stripping can be done using heat combined with stripping agents such as a low pH glycine solution; NaOH solution; a mix of SDS, Tris, and β-mercaptoethanol; or other reagents developed for that purpose.

The order of steps in this workflow can be altered-for example, the cyclic imaging readout of RNA-associated reporters can be done either before or after the cyclic protein readout steps 117-119, or between antigen retrieval 109 and cyclic protein readout. However, for best results, the RNA staining and substrate deposition may be done prior to the cyclic protein readout.

While this disclosure describes specific implementations, these should not be construed as limitations on the scope of the disclosure, but rather as descriptions of features in certain embodiments. Features that are described in the context of separate embodiments can also generally be implemented in combination in a single embodiment. Conversely, various features that are described in the context of a single embodiment can also be implemented in multiple embodiments separately or in any suitable sub-combination. Moreover, although features may be described above as present in certain combinations and even initially claimed as such, one or more features from a claimed combination can generally be excised from the combination, and the claimed combination may be directed to a sub-combination or variation of a sub-combination.

In addition to the embodiments expressly disclosed herein, it will be understood that various modifications to the embodiments described may be made without departing from the spirit and scope of the disclosure. Accordingly, other embodiments are within the scope of the following claims.

What is claimed is:

1. A method, comprising:
(a) contacting a sample with a probe comprising a binding oligonucleotide sequence that hybridizes to at least a portion of an RNA in the sample and a probe identification oligonucleotide sequence associated with the portion of the RNA to which the probe hybridizes;
(b) contacting the sample with a catalytic reporter comprising a recognition oligonucleotide sequence that hybridizes to the probe identification oligonucleotide sequence and a catalytic moiety conjugated to the recognition oligonucleotide sequence;
(c) contacting the sample with an anchoring reporter comprising a substrate conjugated to at least one barcode oligonucleotide sequence, wherein the substrate reacts with the catalytic moiety to deposit the anchoring reporter or a derivative thereof comprising the at least one barcode oligonucleotide sequence in the sample at a location in proximity to the probe;
(d) contacting the sample with a reporter molecule comprising an imaging oligonucleotide sequence conjugated to an optical moiety, wherein the imaging oligonucleotide sequence hybridizes to a barcode oligonucleotide sequence of the at least one barcode oligonucleotide sequence; and
(e) obtaining an image showing light emitted from the optical moiety in the sample.

2. The method of claim 1, further comprising, prior to step (c), removing unhybridized molecules of the catalytic reporter from the sample.

3. The method of claim 1, further comprising, prior to step (d), removing unbound molecules of the anchoring reporter from the sample.

4. The method of claim 1, further comprising repeating steps (b)-(c).

5. The method of claim 4, further comprising repeating steps (d)-(e) to obtain multiple images of the sample.

6. The method of claim 1, further comprising removing or inactivating the optical moiety after the image has been obtained.

7. The method of claim 6, comprising removing the optical moiety by de-hybridizing the imaging oligonucleotide sequence from the barcode oligonucleotide sequence of the at least one barcode oligonucleotide sequence.

8. The method of claim 6, wherein the imaging oligonucleotide sequence is conjugated to an optical moiety through a cleavable linkage, the method comprising cleaving the linkage to remove the optical moiety.

9. The method of claim 8, wherein cleaving the linkage comprises contacting the cleavable linkage with at least one of a chemical agent and an enzymatic agent to cleave the linkage.

10. The method of claim 1, wherein the catalytic moiety comprises an enzyme.

11. The method of claim 10, wherein the enzyme comprises horseradish peroxidase or a derivative thereof that has catalytic activity.

12. The method of claim 1, wherein the substrate comprises at least one member selected from the group consisting of tyramine, a tyramide derivative, hydroxycinnamic acid or a derivative thereof, and styryl-phenol or a derivative thereof.

13. The method of claim 1, wherein at least a portion of the at least one barcode oligonucleotide sequence comprises a double-stranded DNA sequence.

14. The method of claim 1, further comprising, after step (a), contacting the sample with a pre-amplifier, wherein the pre-amplifier comprises:
a first oligonucleotide sequence that hybridizes to the probe identification oligonucleotide sequence; and
at least one second oligonucleotide sequence.

15. The method of claim 14, wherein the recognition oligonucleotide sequence is at least partially complementary to the at least one second oligonucleotide sequence, the method further comprising contacting the sample with the catalytic reporter to bind the catalytic reporter one or more of the at least one second oligonucleotide sequence.

16. The method of claim 15, wherein the at least one second oligonucleotide sequence comprises multiple second oligonucleotide sequences, and wherein multiple catalytic reporters bind to the multiple second oligonucleotide sequences.

17. The method of claim 14, further comprising:
contacting the sample with at least one amplifier,
wherein the amplifier comprises a third oligonucleotide sequence that hybridizes to the at least one second oligonucleotide sequence, and at least one fourth oligonucleotide sequence.

18. The method of claim 17, wherein the recognition oligonucleotide sequence is at least partially complementary to the at least one fourth oligonucleotide sequence, the method further comprising contacting the sample with the catalytic reporter to bind the catalytic reporter one or more of the at least one fourth oligonucleotide sequence.

19. The method of claim 18, wherein the at least one fourth oligonucleotide sequence comprises multiple fourth oligonucleotide sequences, and wherein multiple catalytic reporters bind to the multiple fourth oligonucleotide sequences.

20. A method, comprising:
(a) contacting a sample with a first probe comprising a binding oligonucleotide sequence that hybridizes to a first portion of an RNA in the sample and a first probe identification oligonucleotide sequence associated with the first portion of the RNA, and a second probe comprising a binding oligonucleotide sequence that hybridizes to a second portion of the RNA and a second probe identification oligonucleotide sequence associated with the second portion of the RNA;
(b) contacting the sample with a catalytic reporter comprising a recognition oligonucleotide sequence that hybridizes to both the first and second probe identification oligonucleotide sequences and a catalytic moiety conjugated to the recognition oligonucleotide sequence;
(c) contacting the sample with an anchoring reporter comprising a substrate conjugated to at least one barcode oligonucleotide sequence, wherein the substrate reacts with the catalytic moiety to deposit the anchoring reporter or a derivative thereof comprising the at least one barcode oligonucleotide sequence in the sample at a location in proximity to the first and second probes;
(d) contact the sample with an reporter molecule comprising an imaging oligonucleotide sequence conjugated to an optical moiety, wherein the imaging oligonucleotide sequence hybridizes to a barcode oligonucleotide sequence of the at least one barcode oligonucleotide sequence; and
(e) obtain an image showing light emitted from the optical moiety in the sample.

21. The method of claim 20, further comprising, after step (a), contacting the sample with a pre-amplifier, wherein the pre-amplifier comprises:
a first oligonucleotide sequence that hybridizes to the first and second probe identification oligonucleotide sequences; and
at least one second oligonucleotide sequence.

22. The method of claim 21, wherein the recognition oligonucleotide sequence is at least partially complementary to the at least one second oligonucleotide sequence, the method further comprising contacting the sample with the catalytic reporter to bind the catalytic reporter one or more of the at least one second oligonucleotide sequence.

23. The method of claim 22, wherein the at least one second oligonucleotide sequence comprises multiple second oligonucleotide sequences, and wherein multiple catalytic reporters bind to the multiple second oligonucleotide sequences.

24. The method of claim 21, further comprising:
contacting the sample with at least one amplifier,
wherein the amplifier comprises a third oligonucleotide sequence that hybridizes to the at least one second oligonucleotide sequence, and at least one fourth oligonucleotide sequence.

25. The method of claim 24, wherein the recognition oligonucleotide sequence is at least partially complementary to the at least one fourth oligonucleotide sequence, the method further comprising contacting the sample with the catalytic reporter to bind the catalytic reporter one or more of the at least one fourth oligonucleotide sequence.

26. The method of claim 25, wherein the at least one fourth oligonucleotide sequence comprises multiple fourth oligonucleotide sequences, and wherein multiple catalytic reporters bind to the multiple fourth oligonucleotide sequences.

27. A method, comprising:
(a) contacting a sample with a nucleic acid probe comprising a binding oligonucleotide sequence that hybridizes to at least a portion of an RNA in the sample and a nucleic acid probe identification oligonucleotide sequence associated with the portion of the RNA to which the nucleic acid probe hybridizes;
(b) contacting the sample with a catalytic reporter comprising a recognition oligonucleotide sequence that hybridizes to the nucleic acid probe identification oligonucleotide sequence and a catalytic moiety conjugated to the recognition oligonucleotide sequence;
(c) contacting the sample with an anchoring reporter comprising a substrate conjugated to at least one barcode oligonucleotide sequence, wherein the substrate reacts with the catalytic moiety to deposit the anchoring reporter or a derivative thereof comprising the at least one barcode oligonucleotide sequence in the sample at a location in proximity to the nucleic acid probe;
(d) contact the sample with a first reporter molecule comprising a first imaging oligonucleotide sequence conjugated to a first optical moiety, wherein the first imaging oligonucleotide sequence hybridizes to a barcode oligonucleotide sequence of the at least one barcode oligonucleotide sequence;
(e) obtaining an image showing light emitted from the first optical moiety in the sample;
(f) contacting the sample with a peptide probe comprising a peptide binding moiety conjugated to a peptide probe identification oligonucleotide sequence, wherein the peptide binding moiety binds to a protein or peptide in the sample;
(g) contacting the sample with a second reporter molecule comprising a second imaging oligonucleotide sequence conjugated to a second optical moiety, wherein the second imaging oligonucleotide sequence hybridizes to the antibody probe identification oligonucleotide sequence; and
(h) obtaining an image showing light emitted from the second optical moiety in the sample.

28. The method of claim 27, comprising repeating some or all of steps (a)-(e) with a plurality of different nucleic acid probes to obtain a plurality of images showing light emitted from optical moieties of different first reporter molecules, the emitted light indicating the presence of multiple different RNAs in the sample.

29. The method of claim 27, comprising repeating some or all of steps (f)-(h) with a plurality of different peptide probes to obtain a plurality of images showing light emitted from optical moieties of different second reporter molecules, the emitted light indicating the presence of multiple different proteins and/or peptides in the sample.

30. The method of claim 27, further comprising, after step (f), cross-linking the peptide probe to the sample.

31. The method of claim 27, further comprising, after step (a), contacting the sample with a pre-amplifier, wherein the pre-amplifier comprises:
a first oligonucleotide sequence that hybridizes to the nucleic acid probe identification oligonucleotide sequence; and
at least one second oligonucleotide sequence.

32. The method of claim 31, wherein the recognition oligonucleotide sequence is at least partially complementary to the at least one second oligonucleotide sequence, the method further comprising contacting the sample with the catalytic reporter to bind the catalytic reporter one or more of the at least one second oligonucleotide sequence.

33. The method of claim 32, wherein the at least one second oligonucleotide sequence comprises multiple second oligonucleotide sequences, and wherein multiple catalytic reporters bind to the multiple second oligonucleotide sequences.

34. The method of claim 31, further comprising:
contacting the sample with at least one amplifier,
wherein the amplifier comprises a third oligonucleotide sequence that hybridizes to the at least one second oligonucleotide sequence, and at least one fourth oligonucleotide sequence.

35. The method of claim 34, wherein the recognition oligonucleotide sequence is at least partially complementary to the at least one fourth oligonucleotide sequence, the method further comprising contacting the sample with the catalytic reporter to bind the catalytic reporter one or more of the at least one fourth oligonucleotide sequence.

36. The method of claim 35, wherein the at least one fourth oligonucleotide sequence comprises multiple fourth oligonucleotide sequences, and wherein multiple catalytic reporters bind to the multiple fourth oligonucleotide sequences.

37. The method of claim 27, wherein step (f) is performed after step (d).

38. The method of claim 27, wherein step (f) is performed before step (a).

39. The method of claim 27, wherein the peptide binding moiety comprises an antibody or an antibody fragment.

40. The method of claim 27, wherein the peptide binding moiety comprises a nanobody.

41. The method of claim 27, wherein the peptide binding moiety comprises an aptamer.
